(19) United States
(12) Patent Application Publication
(10) Pub. No.: US 2010/0035335 A1
(43) Pub. Date: Feb. 11, 2010

(54) METAL-ENHANCED FLUORESCENCE FOR THE LABEL-FREE DETECTION OF INTERACTING BIOMOLECULES

(76) Inventors: Joseph R. Lakowicz, Ellicott City, MD (US); Henryk Szmacinski, Ellicott City, MD (US); Krishanu Ray, Ellicott City, MD (US); Mustafa Habib Chowdhury, Washington, DC (US); Kazimierz Nowaczyk, Baltimore, MD (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/537,544

(22) Filed: Aug. 7, 2009

Related U.S. Application Data
(60) Provisional application No. 61/087,585, filed on Aug. 8, 2008.

(51) Int. Cl. C12M 1/34; G01N 21/64

(52) U.S. Cl. 435/287.1; 422/82.08; 977/773

(57) ABSTRACT

A method for enhancing fluorescence of a biomolecule includes the step of associating the biomolecule having intrinsic fluorescence with a sensing surface that contains nanostructured metal. Association of the biomolecule with the nanostructured metal enhances its intrinsic fluorescence, which is detected upon exposure to electromagnetic radiation of a suitable wavelength. The sensing surface may include capture or ligand molecule which binds to the biomolecule and sequesters it in proximity to the nanostructured metal, thereby causing its fluorescent signal to be enhanced. The method can be used in label-free bioassays for detection of interacting biomolecules, such as antibody-antigen binding.

### CROSS-REFERENCE TO RELATED APPLICATIONS

This application claims benefit of U.S. provisional application No. 61/087,585 filed Aug. 8, 2008 titled “Application of metal-enhanced fluorescence to label-free detection of interacting biomolecules.”

### STATEMENT OF FEDERALLY SPONSORED RESEARCH AND DEVELOPMENT

This invention was made with government support under Grant Numbers EB006521 and HG002655 awarded by the National Institutes of Health. The government has certain rights in the invention.

### BACKGROUND OF THE INVENTION

1. Field of the Invention

The invention generally relates to methods for label-free enhancement of the intrinsic fluorescence of biomolecules. In particular, the invention provides label-free methods that enhance intrinsic fluorescence of biomolecules by associating the biomolecules with nanostructured metals.

2. Background of the Invention

Fluorescence detection presently is a central technology in the biosciences. The applications of fluorescence include cell imaging, medical diagnostics and biophysical research. Another growing use of fluorescence is for measurements of a large number of samples as occur on DNA arrays, protein arrays and high throughput screening (HTS). HTS typically includes testing of a large number of small molecules for biological activity, most often drug-receptor interactions. Almost all the applications of fluorescence require the use of labeled drugs and labeled biomolecules, which becomes increasingly inconvenient as the number of compounds to be tested has increased. The need for labeling of the biomolecules with extrinsic fluorophores results in increased costs and complexity. Because of this added complexity, there is a rapidly growing interest in the development of methods for sensitive, label-free detection (LFD) of biomolecules and their interactions.

### SUMMARY OF THE INVENTION

The present invention provides metallic nanostructures that enhance the intrinsic fluorescence of biomolecules when they are placed in proximity to the nanostructures. This discovery makes possible the development of sensitive assays which do not require the use of extrinsic labels, thereby reducing the cost and complexity of the assays. Further, the elimination of extrinsic labels decreases artifacts that can be caused by labeling. Biomolecules that are particularly suited for analysis by the methods of the invention include proteins and nucleic acids.

The invention provides a label-free method of detecting a biomolecule that has intrinsic fluorescence, comprising the steps of providing a sensing surface comprising at least one nanostructured metal; contacting the sensing surface with a composition; interrogating the sensing surface with electromagnetic radiation; and detecting the presence or absence of said biomolecule in the composition from changes in intrinsic fluorescence of the biomolecule that occur when the biomolecule is in close proximity to the nanostructured metals on the sensing surface. In some embodiments, the nanostructured metals are deposited on a substrate, which may be a dielectric substrate, for example, silica, silicon, quartz, plastic, silicon nitride, metallic oxides with either no or low fluorescence in the UV wavelength range, and glass. In some embodiments, the substrate transmits electromagnetic radiation therethrough. The substrate may be a well, a plate, a tube, a wire, or a bead, and the nanostructured metal may includes one or more of aluminum, silver, platinum and gold. The nanostructured metal:may be in mart such as a nanostructured film, a metal colloid, one or more nanoparticles deposited on a support, a nanoparticle dimer, a nanoparticle cluster, a nanoparticle array on a support, patterned nanoholes in a layer of continuous metal film, and nanometallic islands. The nanostructured metal may be present on the substrate in the form of a patterned array of metal nanoparticles, nanoholes, or metal surfaces. In some embodiments, the sensing surface includes a grating which is part of one or more of the substrate and the at least one nanostructured metal. The method may further comprise use of a capture molecule associated with the sensing surface which binds to or interacts with the biomolecule. The capture molecule may be, for example, an antibody or antigen, and may include one or more of proteins, peptides, nucleic acids, carbohydrates, cofactors, metal ions, small molecule candidates, enzyme substrates, inhibitors, agonists, antagonists, and ligands. Further, in some embodiments, the capture molecule is associated with the sensing surface using a linking moiety which spaces the capture molecule away from the substrate. After binding or interacting with the biomolecule, the capture molecule may maintain, for a period of time, the biomolecule in close proximity (e.g. between 2 to 50 nm) to the at least one nano-structured metal. The at least one nanostructured metal may include one or more of aluminum, silver, platinum and gold, and may be in a form selected from a grating, a nanostructured film, a metal colloid, one or more nanoparticles deposited on a support, a nanoparticle dimer, a nanoparticle cluster, a nanoparticle array on a support, and patterned nanoholes in a layer of continuous metal film. The at least one nanostructured metal includes nanoparticles may be of a size ranging from 20 nm to 100 nm. In particular, nanostructured silver particles may range in size from 40 nm to 100 nm, and nanostructured aluminum particles may range from 40 nm to 100 nm. Further, the at least one nanostructured metal may include metallic islands of a subwavelength size of up to 500 nm. In some embodiments, the biomolecule includes one or more of N-acetyl-L-tryophanimide and N-acetyl L-tyrosinamide, or residues of N-acetyl-L-tryophanimide and N-acetyl-L-tyrosinamide, or one or more of tryptophan, tyrosine, and phenylalanine molecules. In other embodiments, the biomolecule is selected from proteins, peptides and nucleic acids.

In some embodiments of the method, the step of interrogating is performed using electromagnetic radiation of a wavelength ranging from 250 to 320 nm. The step of interrogating may be performed by one or more of illumination of a top surface of the sensing surface, illumination of a bottom surface of the sensing surface through a substrate, use of evanescent wave using total internal reflection, and excitation at a surface plasmon angle. Further, the step of detecting may be carried out by measuring fluorescence intensity using a detector positioned to detect emissions from a location selected from a top surface of said sensing surface, a bottom surface of said sensing surface through a substrate, and a plasmon resonance angle for emission. Alternatively, the step of detecting may be carried out by collecting emissions using a collection enhancement mechanism that enhances collection efficiency by selective directionality of fluorescent light. The collection enhancement mechanism may include one or more of dielectric arrays and metallic arrays. The step of interrogating and the step of detecting may be performed using a source of electromagnetic radiation and a detector on opposite sides of the sensing surface. The steps of interrogating and detecting may be performed using a source of electromagnetic radiation and a detector on the same side of the sensing surface. The step of detecting may be performed without prior washing of the sensing surface, and may be performed in the presence of a sample solution. The method may further comprise the step of using a fluorescence signal detected in the detecting step to determine an amount of the biomolecule which is in close proximity to the nanostructured metals on the sensing surface.

The invention also provides a label-free detecting system for detecting a biomolecule that has intrinsic fluorescence, comprising: a sensing surface comprising at least one nanostructured metal; a source of electromagnetic radiation that is used to interrogate said sensing surface; and a detector for detecting the presence or absence of said biomolecule in a composition based on changes in intrinsic fluorescence of said biomolecule that occur when said biomolecule is in close proximity to said nanostructured metal on said sensing surface. In some embodiments, the system also comprises a substrate, the at least one nanostructured metal being deposited on the substrate, e.g. a dielectric substrate made from silica, silicon, quartz, plastic, silicon nitride, metallic oxides with either no or low fluorescence in the UV wavelength range, or glass. In some embodiments, the substrate transmits electromagnetic radiation therethrough. The substrate may be a well, a plate, a tube, a wire, and a bead. The at least one nanostructured metal may include one or more of aluminum, silver, platinum and gold, and may be in a form such as a nanostructured film, a metal colloid, one or more nanoparticles deposited on a support, a nanoparticle dimer, a nanoparticle cluster, a nanoparticle array on a support, and patterned nanoholes in a layer of continuous metal film. The at least one nanostructured metal may be present on the substrate in the form of a patterned array of metal nanoparticles, nanoholes, or metal surfaces. The sensing surface may include a grating which is part, of one or more of the substrate and the at least one nanostructured metal. The system may further comprise a capture molecule associated with the sensing surface which binds to or interacts with the biomolecule. The capture molecule may be an antibody or antigen, or may be or include one or more of proteins, peptides, nucleic acids, carbohydrates, cofactors, metal ions, small molecule candidates, enzyme substrates, inhibitors, agonists, antagonists, and ligands. The capture molecule may be associated with the sensing surface using a linking moiety which spaces the capture molecule away from the substrate, and, after binding or interacting with the biomolecule, may maintain, for a period of time, the biomolecule in close proximity (e.g. between 2 to 50 nm) to the at least one nano-structured metal. The at least one nanostructured metal may include one or more of aluminum, silver, platinum and gold, and may be in a form selected from a grating, a nanostructured film, a metal colloid, one or more nanoparticles deposited on a support, a nanoparticle dimer, a nanoparticle cluster, a nanoparticle array on a support, a and patterned nanoholes in a layer of continuous metal film. The at least one nanostructured metal includes nanoparticles of a size ranging from 20 nm to 100 nm. In particular, nanostructured silver particles may range in size from 40 nm n to 100 nm, and nanostructured aluminum particles may range from 40 nm to 100 nm, and may include metallic islands of a subwavelength size of up to 500 mm.

In some embodiments of the method, the step of interrogating is performed using electromagnetic radiation of a wavelength ranging from 250 to 320 nm. In some embodiments of the invention, the source of electromagnetic radiation provides for one or more of illumination of a top surface of the sensing surface, illumination of a bottom surface of the sensing surface through a substrate, use of evanescent wave using total internal reflection, and excitation at a surface plasmon angle. The detector may be positioned to detect emissions from a location selected from a top surface of the sensing surface, a bottom surface of the sensing surface through a substrate, and a plasmon resonance angle for emission. The system may further comprise a collection enhancement mechanism that enhances collection efficiency by selective directionality of fluorescent light. The collection enhancement mechanism may include one or more of dielectric arrays and metallic arrays. A source of electromagnetic radiation and the detector may be positioned on opposite sides or on the same side of the sensing surface. The system may further comprise a means for using a fluorescence signal detected in the detecting step to determine an amount of the biomolecule which is in close proximity to said nanostructured metals on the sensing surface.

The invention further provides a probe for label free detecting of a biomolecule that has intrinsic fluorescence, comprising: a substrate; a sensing surface comprising at least one nanostructured metal positioned on the substrate; and a capture molecule associated with the sensing surface which binds to or interacts with the biomolecule. The substrate may be a dielectric substrate of, for example, silica, silicon, quartz, plastic, silicon nitride, metallic oxides with either no or low fluorescence in the UV wavelength range, or glass. The substrate may transmit electromagnetic radiation therethrough, and may be a well, a plate, a tube, a wire, or a bead. The at least one nanostructured metal may include one or more of aluminum, silver, platinum and gold, and may be in a form such as a nanostructured film, a metal colloid, one or more nanoparticles deposited on a support, a nanoparticle dimer, a nanoparticle cluster, a nanoparticle array on a support, or patterned nanoholes in a layer of continuous metal film. In addition, the at least one nanostructured metal may be present on the substrate in the form of a patterned array of metal nanoparticles, nanoholes, or metal surfaces. The sensing surface may include a grating which is part of one or more of the substrate and the at least one nanostructured metal. The capture molecule may be an antibody or antigen, or may be or include one or more of proteins, peptides, nucleic acids, carbohydrates, cofactors, metal ions, small molecule candidates, enzyme substrates, inhibitors, agonists, antagonists, and ligands. The capture molecule may be associated with the sensing surface using a linking moiety which spaces the capture molecule away from the substrate, and, after binding or interacting with the biomolecule, may maintain, for a period of time, the biomolecule in close proximity (e.g, between 2 to 50 nm) to the at least one nano-structured metal. The at least one nanostructured metal includes nanoparticles of a size ranging from 20 nm to 100 nm. In particular, nanostructured silver particles may range in size from 40 nm to 100 nm, and nanostructured aluminum articles may range from 40 nm to 100 nm, and may include metallic islands of a subwavelength size of up to 500 nm. In some embodiments of the method, the step of interrogating is performed using electromagnetic radiation of a wavelength ranging from 250 to 320 n.

The invention further provides a method for enhancing the fluorescence of a fluorophore in an ultraviolet (UV)-blue region or in a UV region of a spectrum, or both. The method comprises the step of associating the fluorophore with a nanostructured metal. The UV-blue region corresponds to wavelengths from 240 to 400 nm; the UV region corresponds to wavelengths from 250 to 380 nm.

### BRIEF DESCRIPTION OF THE DRAWINGS

FIG. 1A-B. Exemplary device and chemical structures of the probes, A, schematic representation of an embodiment of the invention; B, schematic of the sample geometry.

FIG. 2A-D. A, Schematic diagram of the model radiating fluorophore/metal nanoparticle system studied using Finite-Difference Time-Domain (FDTD); Near-field intensity distribution around; B, a 500 aluminum nanoparticle separated 10 nm from a fluorophore radiating at 375 nm and oriented along the x-axis, C, near-field intensity distribution around the isolated fluorophore; and D, near-field enhancement around the aluminum particle. Note all images are displayed in the log scale.

FIG. 3. Control calculation showing the radiated power enhancement for an isolated dipole oriented both in the perpendicular direction and parallel direction.

FIG. 4. Intensity of avidin spotted on the bare quartz and on the SIFs with sequentially increased concentration per spot.

FIG. 5. Intensity decays of avidin spotted on bare quartz (3 layers of avidin) and on SIFs.

FIG. 6. Absorption spectrum of avidin in aqueous solution (1) and emission spectra of avidin measured on the SIFs (2) and quartz (3) after spotted from aqueous solution and dried (approximately 3 avidin layers), The absorption spectrum of SIFs is also shown (dashed line).

FIG. 7A-B. Emission spectra of (BSA-Avidin) layers deposited on quartz (A) and on SIFs (B).

FIG. 8. Intensity decay of avidin in buffer solution and multi-layers deposited on quartz surface and SIFs via a layer of bovine serum albumin (BSA)biotin. The multi-layer consisted of three BSA-Bt-avidin layers. The lifetime value in solution is for avidin in phosphate buffer.

FIG. 9, Radiative power spectrum of 100 silver nanoparticle separated 8 nm from a radiating dipole calculated by FDTD method. Emission spectra of NATA and NATA-tyr on SIFs are also included.

FIG. 10A-D. A, Schematic diagram of the model radiating fluorophore/metal nanoparticle system studied using FDTD; Near-field intensity distribution around B, a 100 silver nanoparticle separated 8 nm from a probe radiating at 350 nm and oriented along the x-axis, C, near-field intensity distribution around the isolated probe and D, near-field enhancement around the silver nano-particle. Note all images are displayed in the log scale.

FIG. 11A-D. A, Schematic diagram of the model radiating fluorophore/metal nanoparticle system studied using FDTD; Near-field intensity distribution around B, a 100 silver nanoparticle separated 8 nm from a probe radiating at 305 nm and oriented along the x-axis, C, near-field intensity distribution around the isolated probe and D, near-field enhancement around the silver nano-particle. Note all images are displayed in the log scale.

### DETAILED DESCRIPTION

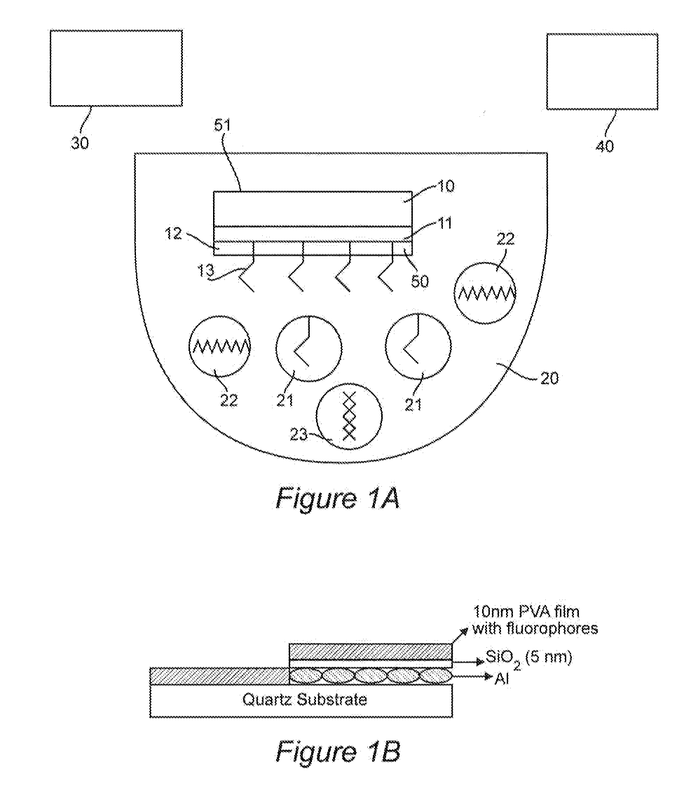

A schematic representation of an exemplary device of the invention is shown in FIG. 1A, Shown in FIG. 1A is substrate 10 (which is shown as a rectangle but may also be in the form of a tube, bead, wire, etc.); and nanostructured metal 11 (e.g. aluminum, silver, gold, platinum, etc.,) which may be in the form of a layer or multiple layers and may or may not be continuous on substrate 10. Nanostructured metal 11 may be in the form of particles, films, etc., as described in detail below, and may be patterned (e.g. as a grid, lines, etc.) or not patterned. Also shown is optional layer 12, which may represent a layer of material such as SiO₂ positioned over nanostructured metal 11 to protect the metal, or may represent linker or spacer moieties to which capture molecules 13 are attached. If layer 12 is absent, capture molecules 13 may be attached directly to nanostructured metal 11. The portion of the device with which the capture molecules 13 are associated may also be referred to as a sensing surface. According to the invention, when the device is exposed to sample 20, which contains a mixture of molecules of types 21, 22, and 23, molecules capable of binding to capture molecules 13 (in this case, molecules 21) will bind to and be retained on the device. One or both of capture molecules 13 or molecules 21 that are retained possess intrinsic fluorescence. This intrinsic fluorescence is enhanced and thus readily detectable by proximity to nanostructured metal 11. For example, if capture molecules 13 are not fluorescent but molecules 21 are intrinsically fluorescent, then an enhanced fluorescent signal will be produced by the binding of molecules 21 to capture molecules 13. If capture molecules 13 are intrinsically fluorescent, the binding of molecule 21 may effect a change in that intrinsic fluorescence, which is detected by the device. If both capture molecules 13 and molecules 21 are intrinsically fluorescent, changes upon binding in the detectable fluorescence of one or the other or both are detected. In order to produce fluorescence, the intrinsically fluorescent molecules are exposed to (i.e. interrogated by or with) a source of electromagnetic radiation 30 in order to excite the molecule. In one embodiment of the invention, the electromagnetic radiation is in the UV range, e.g. in the range of from about 250 to about 380 nm, EM source 30 may be positioned so as to deliver direct illumination to a top surface 50 of the device; or to a bottom (back) surface 51 of the device (i.e. through substrate 20); or by evanescent waves using total internal reflection (TIR); or by excitation at one or more surface plasmon angles. Fluorescence is detected by detector 40, which may be positioned to detect fluorescence emanating from fluorophores associated with top surface 50, or fluorescence that passes through substrate 10 and out from bottom surface 51, or fluorescence from a plasmon resonance angle. Fluorescence emissions may be collected using, for example, dielectric arrays and/or metallic arrays with features that enhance the collection of efficiency by, for example selective directionality of the fluorescent light. Emissions are typically in the 260 to 500 nm range.

In another aspect, the invention provides methods for enhancing the ultraviolet (UV) region (or alternatively, the UV-blue region) fluorescence of any fluorophore. The method involves associating the fluorophore with a nanostructured metal. By “UV-blue region” we mean the region of the spectrum corresponding to from about 240 to about 400 nm. By “UV region” we mean the region of the spectrum corresponding to from about 250 to about 380 nm.

As described herein, intrinsic fluorescence of biomolecules is enhanced by proximity to nanostructured metals. This provides the ability to develop assays to monitor the behavior of biomolecules by measuring their intrinsic fluorescence without the use of extrinsic labels. In order to do so, the fluorescent biomolecule is associated with or attached to a nanostructured metal. In one aspect of the invention, the enhancement is particularly in the UV-blue region of the spectrum. Enhancement of fluorescence may include increases in emission intensity (and other spectral changes), increasing the rate of excitation, increases in radiative decay and shortening of the fluorescence lifetime, which may result in the capability for increased cycling of fluorescent measurements, and allows detection of, e.g. specific proteins. Generally, the measurable intrinsic fluorescence of the biomolecule is enhanced or increased by at least about 2-fold or more (i.e. 2× or more) of the level of fluorescence that is measured when the biomolecule is not associated with a nanostructured metal.

By “associated with” or “attached to” we mean that the biomolecule is retained on or in close proximity to the metal surface by chemical bonding. For example, depending on the type of molecule and the type of metal, the molecule may be held to the metal by electrostatic interactions, covalent bonding, hydrophobic interactions or specific biological interactions, etc.). Further, bonding to the surface can be the result of biological interactions such as protein-protein binding, protein-nucleic acid binding and similar effects. Further, in some embodiments of the invention, the fluorescent biomolecule may be attached to the metal surface indirectly by means of a linker or spacer molecule (which may or may not be fluorescent) to which the biomolecule of interest (i.e. that for which changes in fluorescence are to be measured) is attached. In this manner, the precise distance of the biomolecule from the surface of the metal can be varied or adjusted or “tuned” to modulate the fluorescent signal that is obtained. Also, this may make the biomolecule more sterically accessible to other entities of interest, e.g. to potential ligands, during an assay. Generally, the distance of fluorophores within a molecule will be at a distance in the range of from about 1 to 100 nm, and preferably in a range of from about 2 to about 10, 20, 30, 40 or 50 nm. Fluorescence is enhanced the most by particles when the fluorophore is at a distance of from about 5 to about 20 nm. With other types of surfaces, (e.g. films) the range of distance is from about 5 to about 70 nm. The size of, for example, an antibody is about 8 m/z, so even multiple layers of antibodies (or other molecules) will be within the range of influence of the nanostructured metal. The methods of the invention can enhance intrinsic fluorescence of molecules possessing fluorophores located at these distances, whether the fluorescent moiety is located within a molecule that is attached directly to the nanostructured metal, or within a molecule that is attached to the directly attached molecule. In other words, the invention also encompasses detection of changes in intrinsic fluorescence in layers of associated molecules.

By “nanostructured metal” we mean that the metal structure contains features with dimensions that are in the nanometer range, i.e. in the range of from about 1 to about 1000 nm, and preferably in the range of from about 10 to about 500 nm, and more preferably in the range of from about 20 nm to about 400 nm. In some cases, the metal can be a smooth surface. In other cases, the metal can have regular or random features in the form of particles, particle clusters or holes. The overall dimensions of a single sensing surface can range from a single particle or hole to extended arrays of features. Typically, the specific sensing regions will be smaller than 1 cm×1 cm, allowing multiple assays on a single structure. Generally, the metals are deposited on the surface of a substrate, for example, in the form of a film or coating with a depth in the nanometer range (preferably, for example, from about 2 nm to about 500 nm, or more preferably in the range of from about 4 nm to about 300 nm). The film may be comprised of one layer or of several layers of metal that are deposited one upon another. The film may be either continuous or discontinuous e.g. so-called island films or islands of particles may be formed on the substrate, depending on the amount of metal that is deposited and/or the method of deposition, etc. Alternatively, various shapes of films may be made, e.g. strips, circles, dots, lines, gratings, nanohole arrays, various structures that provide directional radiation, or other suitable shapes, by, for example, masking portions of the substrate during metal deposition or by other techniques. If multiple separate areas of metal film are located on a substrate, the same or different biomolecules may be attached to each area. Those of skill in the art will recognize that the films themselves are generally made up of a plurality of metal, particles with dimensions in the nanometer range. By controlling the conditions of deposition, and the type of metal, the size of the particles can be adjusted, and this will influence the properties of the film, for example, the thickness, contour (e.g. roughness, etc.), topography, and other properties of the film. Further, in some embodiment, the metal is not deposited as a film but is in a particulate state, and the biomolecules are attached to or interact with the particles. The particles may be in suspension, in the form of a colloid, as nanoparticle dimers, or as nanoparticle clusters. For example, the metal colloids can be suspended in a liquid solution and in this case a bioactive layer may be on the colloids so that a biomolecule from a sample binds to one or more moieties in the bioactive layer. Alternatively, colloids may be deposited from solution to a solid substrate surface. In this case, the substrate will contain a uniform size of metallic particles. All of such metallic nanostructures are useful in the practice of the invention.

The substrates on which the nanostructured metals are disposed may be any of several suitable types and shapes which are amenable to the association of biomolecules with the nanostructured metals deposited thereon. Examples include but are not limited to: various substantially flat surfaces; the wells of multi-well assay plates; various beads; tubes (either external or internal); wires; microscope slides, cover slips, specially designed surfaces and shapes for specific applications, etc. The substrates may be formed from any material that is suitable for metal deposition, and for use in the assay methods disclosed herein, for example, quartz, various stable plastics, polymers and chromatography media, etc. For example, the metal particles may be on or embedded into polyacrylamide gels, which may be used as slabs or in tubes.

In general, the substrates on which the nanostructured metals are deposited will be dielectric substrates, examples of which include but are not limited to silica, silicon, quartz, various plastics, etc. However, in some embodiments, the substrate may be made of a non-dielectric material (such as a metal) that has a dielectric coating. Nanostructured metals may then be deposited on the dielectric coating.

Examples of biomolecules which possess intrinsic fluorescence that is enhanced by the methods of the invention include but are not limited to those which contain the amino acid residues tryptophan (Trp), tyrosine (Tyr) and phenylalanine (Phe). This group of biomolecules generally includes proteins (e.g. receptors, enzymes, etc.), polypeptides and peptides, as well as various hybrid molecules which contain one or more fluorescent amino acids, e.g. RNA-peptide hybrids, DNA-peptide hybrids, etc. Nucleic acids such as DNA, RNA and hybrids thereof also possess intrinsic fluorescent properties. Other biological molecules that have intrinsic fluorescence include but are not limited to bilirubin, which is highly fluorescent when bound to a specific site on serum albumin; and zinc protoporphyrin, which is formed in developing red blood cells instead of hemoglobin when iron is unavailable or lead is present, and which has a bright fluorescence. Other biomolecules that possess intrinsic fluorescence will occur to those of skill in the art and are intended to be encompassed by the present invention. Further, more than one type of molecule or biomolecule may be attached to a metal surface.

Those of skill in the art will recognize that, in the practice of the invention, such intrinsically fluorescent molecules may either be associated with the nanostructured metals, or may bind to another molecule (e.g. a capture or ligand molecule) that is associated with the nanostructured metals.

The invention may be practiced using molecules that include any type of fluorescent moiety. Exemplary fluorescent moieties include but are not limited to various aromatic ring systems (e.g, benzene-like), various heterocyclics (e.g. pyridine), 2-ring compounds such as napthalene and indole, various 3-ring compounds, etc. Examples of such compounds include but are not limited to molecules of the DNS class such as 1-1-dimethylamino-5-naphthalenesulfonic acid, the ANS class such as 1-anilino-8-naphthalenesulfonic acid, and reactive derivatives of the same such as the sulfonly chlorides. Other examples are coumarin, carbazole, and stilbene, and derivatives of these molecules.

Metals that may be used in the practice of the invention include but are not limited to silver, gold, aluminum, platinum, etc. Further alloys or other types of mixtures of these may also be employed. For example, colloids can be formed from mixtures of silver and gold.

Silver particles may be coated with a gold layer. Further, the surface of one metal can be coated with a different metal. These coatings may be used to modify the surface reactivity or optical properties of the method.

In one embodiment of the invention, an assay device is formed by deposition of a nanostructured film of metal onto a suitable substrate. Thereafter, one or more types of biomolecules with intrinsic fluorescence is/are attached to the metal layer, e.g. by electrostatic interactions. The device may then be used to assay or monitor changes in the fluorescence of the biomolecule in response to changes in its environment. Such changes may be caused by a change in the folded conformation of the biomolecule or of the positioning or orientation of the fluorescent moiety in the biomolecule. However, the change in fluorescence may also be due to other factors such as increased or decreased quenching, to etc. Changes that might effect the fluorescence of the biomolecule include but are not limited to changes in the composition or properties of media to which the assay device is exposed, such as changes in pH, in ionic strength, temperature, hydrophilicity/hydrophobicity, buffering agents, etc. In addition, changes include the interaction of the biomolecule with other molecular entities in the environment, particularly those which are not bound to the metal surface but which are present in surrounding media, e.g. binding of ligands such as proteins or peptides, carbohydrates, nucleic acids, metals/metal ions, various cofactors, enzyme substrates, various small molecules e.g. designer drugs, various inhibitors, etc. In particular, devices of the invention are useful in high throughput screening of multiple entities such as small molecule drug candidates. In some cases, the biomolecule of interest is attached to the device and exposed to the drug candidates, and changes in fluorescence that occur as a result of exposure may be taken as indicative of an interaction such as binding of the drug candidate by the biomolecule, Conversely, the absence of changes in fluorescence may be interpreted as a lack of interaction between the biomolecule and the drug.

Alternatively, especially in cases where the candidate is a small molecule (e.g. Mr less than about 500D), one or more candidate drugs may be immobilized or attached to the metal nanostructure and then exposed to a fluorescent biomolecule of interest such as a protein. Binding of the fluorescent biomolecule to a candidate drug brings the biomolecule into close proximity to the nanostructured metal. Fluorescence of the bound biomolecule is then enhanced and detected only on areas of the device where small molecules that can successfully bind the florescent biomolecule are present. Examples of molecules that may be bound to the nanostructured metal include but are not limited to aptamers (e.g. nucleic acid or peptide molecules that bind a specific target molecule), antibodies, etc.

In other embodiments of the invention, molecules bound to the nanostructured metal may not be intrinsically fluorescent, but they may be used to capture or bind to molecules that are intrinsically fluorescent. Alternatively, both a molecule attached to the nanostructured metal and a molecule that interacts with an attached molecule may possess intrinsic fluorescence. In some embodiments, neither molecule may possess intrinsic fluorescence until they are bound to each other, thereby generating fluorescence that is enhanced by being in proximity to the nanostructured metal. In other words, the binding of a molecule that is attached to a nonstructured metal to another molecular entity may create a fluorophore.

One exemplary use of the invention is to investigate biomolecules. By “biomolecules” we mean those molecules that are typically associate d with living organisms, e.g. proteins, polypeptides, nucleic acids, lipids, carbohydrates, and combinations thereof. However, those of skill in the art will recognize that many other organic and non-organic molecules exist that may be detected or assayed by the methods of the invention. Some are related to biological systems (e.g. various co-factors, metal ions, etc.). Others may be artificial i.e. synthetic fluorescent or non-fluorescent molecules, e.g. molecules that are drug candidates, or molecules that may have other useful properties. For example, the present invention is useful for screening or identifying molecules that bind to or otherwise interact with molecules attached to metal nanostructures. Screened molecules may be candidate drugs, various fluorophores, various inhibitors or activators, single strand nucleic acids, etc.

Other permutations of such assays and assay devices will occur to those of skill in the art, and all are intended to be encompassed by the present invention.

In some embodiments of the invention, measurements of the changes in fluorescence may be equated with the amount of an entity that binds to the biomolecule. In other embodiments, the technique may be used to sequester and purify the entities that bind to the biomolecule.

In further embodiments of the invention, coatings of non-metallic substances may be interposed between the metal nanostructure and the biomolecule, the biomolecule being attached to the non-metal layer. Alternatively, a biomolecule with intrinsic fluorescence may be attached to a metal nanostructure and then additional coatings or layers of other substances may be deposited on the biomolecule Examples of such layers include but are not limited to e.g. SiO₂, other biomolecules, etc.

The following examples serve to illustrate the invention but should not be construed as limiting in any way.

### EXAMPLES

### Example 1

Aluminum Nano-structured Films as Substrates for Enhanced Fluorescence in the Ultraviolet—Blue Spectral Region

Particulate aluminum films of varied thicknesses were deposited on quartz substrates by thermal evaporation. These nano-structured substrates were characterized by scanning electron microscopy (SEM). With the increase of aluminum thickness, the films progress from particulate towards smooth surfaces as observed by SEM images. To date, metal-enhanced fluorescence (MEF) has primarily been observed in the visible-NIR wavelength region using silver or gold island films or roughened surfaces. We now show that fluorescence could also be enhanced in the ultraviolet-blue region of the spectrum using nano-structured aluminum films. Two probes, one in the ultraviolet and another one in blue spectral region have been chosen for the present study. We observed increased emission, decrease in fluorescence lifetime and increase in photostability of a DNA base analogue 2-aminopurine (2-AP) and a coumarin derivative (7-HC) in 10 nm spin-casted polyvinyl alcohol film on Al nanostructured surfaces. The fluorescence enhancement factor depends on the thickness of the Al films as the size of the nanostructures formed varies with Al thickness. Both probes showed increased photostability near aluminum nano-structured substrates, which is consistent with the shorter lifetime. Our studies indicate that Al nano-structured substrates can potentially find widespread use in MEF applications particularly in the UV-blue spectral region. Further more, these Al nano-structured substrates are very stable in buffers that contain chloride salts compared to usual silver colloid based substrates for MEF, thus furthering the usefulness of these Al based substrates in many biological assays where high concentration of salts are required. Finite-Difference Time-Domain (FDTD) calculations were also employed to study the enhanced near-fields induced around aluminum nanoparticles by a radiating fluorophore, and the effect of such enhanced fields on the fluorescence enhancement observed was discussed.

### Introduction

Fluorescence detection presently is a central technology in the biological research and medical diagnostic. While fluorescence is a sensitive method there is a continuing need for increase sensitivity, as evidenced by the use of amplification methods such as ELISA^{1,2} and PCR^{3,4}. However, the detection and sensitivity, in general is limited by the fluorescence quantum yield and photostability of the probe and additionally autofluorescence from the sample. During the past five years there has been a growing interest in the use of colloidal metal particles or roughened metal surfaces for enhanced fluorescence⁵⁻¹¹, in particular silver colloids and silver island films (SIFs). In recent years we have reported on the favorable effects of silver nanoparticles deposited randomly on glass substrates (primarily in the form of SIFs) for increasing the emission intensities (quantum yields), reducing fluorescence lifetimes and increasing the photostability of fluorophores with visible excitation and emission wavelengths. A large number of reports have demonstrated that fluorescence intensities can be increased 10-fold or more when the fluorophores are in close proximity to the silver particles.^{8,11,12} The mechanism of this enhancement is, at least in part, due to an increased rate of radiative decay near the particles. Fluorophores in the excited state undergo near-field interactions with the metal particles to create plasmons. While the mechanism of metal-enhanced fluorescence (MEF) is not completely understood, an important factor is the ability of the plasmons to radiate away from the particle.¹²

The remarkable optical characteristics of metallic nano-structures/particles observed of varying sizes, encompassing the visible range, have been investigated by various far-field optical spectroscopy techniques as well as near-field imaging spectroscopy.¹³⁻¹⁷ When considering aluminum for MEF, it was reported that aluminum quenches fluorescence. However, it has been shown that excited fluorophores over smooth aluminum film could create surface plasmon which eventually radiate into the substrate.¹⁹ The spectral properties of the radiation were found to be essentially identical to those of the fluorophore, except for a highly p-polarized emission. The angular dependence of the radiation, as well as the p-polarization, are consistent with radiating surface plasmons. According to the radiating plasmons (RP) model for MEF, the scattering component of metal particle extinction contributes to MEF and the absorption component contributes to quenching. So far, MEF has primarily been reported for silver and gold surfaces. This choice of metals restricts the selection of fluorophores to hose absorbing and emitting at visible or longer wavelengths. However, many widely used fluorophores absorb or emit at ultraviolet wavelengths. In this regard, aluminum nano-structured substrates can potentially find widespread use in MEF applications particularly in the UV-blue spectral regime. Aluminum is also less expensive than gold or silver. Moreover, aluminum nano-structured substrates are very stable in buffers that contain chloride salts compared to usual silver colloid based substrates for MEF, thus furthering the usefulness of these aluminum based substrates in many biological assays where high concentration of salts are required.

In this Example, we describe studies of the MEF displayed by fluorophores in close proximity to particulate aluminum films. These particulate Al surfaces were obtained by thermal evaporation followed by depositing a layer of SiO₂. The silica layer was used as spacer layer to avoid direct contact of the fluorophore with the aluminum surfaces. It is reported that the extent of MEF depends on the size of the particles and the distance between the metal and the fluorophore^{5,11,12}. Accordingly, we investigated the variation in the size of the aluminum nanostructures to identify the optimum conditions for MEF in the ultra-violet and blue spectral region. We also used numerical calculations in the form of the Finite-Difference Time-Domain (FDTD) technique to understand our experimental results. FDTD is an implementation of Maxwell's time-dependent curl equations for solving the temporal variation of electromagnetic waves within a finite space that contains a target of arbitrary shape, and has recently become the state-of-the-art method for solving Maxwell's equations for complex geometries.²⁰⁻²⁶ A major advantage of FDTD is that it is a direct time and space solution, and hence offers the user a unique insight into a myriad of problems in photonics. More information on the FDTD technique can be found in Refs.²⁰⁻²⁶ Our calculations reveal that fluorophores in the near-field of an aluminum nanoparticle induces enhancements of the near-fields around the particle, which we believe play a role in our observation of MEF from aluminum surfaces.

### Material and Methods:

Aluminum slugs, silicon monoxide, and 2-aminopurine (2-AP) were purchased from Sigma-Aldrich and used as received. 7-hydroxycoumarin-3-carboxylic acid, succinimidyl ester (7-HC) was obtained from Invitrogen Molecular Probes. Distilled water (with a resistivity of 18.2 MΩ-cm) purified using Millipore Milli-Q gradient system was used for sample preparation, Aluminum was deposited on quartz slides using an Edwards Auto 306 Vacuum Evaporation chamber under high vacuum (<5×10⁻⁷ Tort). In each case, the metal deposition step was followed by the deposition of 5 nm of silica via evaporation without breaking vacuum. This step served to protect the metal surface as well as it adds a spacer layer between the metal surface and fluorophore. The deposition rate was adjusted by the filament current and the thickness of film was measured with a quartz crystal microbalance. Fluorophores were deposited on the surface of the substrate by spin coating (Specialty coating system Inc., Speedline Technologies, Ind.) 0.25 wt % aqueous solution of low molecular weight poly(vinyl alcohol) (PVA, MW. 13 000-23 000; Aldrich) at 3000 rpm. This composition of PVA forms ˜10 nm thick film and is schematically depicted in FIG. 1B.

Absorption spectra were collected using a Hewlett-Packard 8453 spectrophotometer. Fluorescence spectra of probes on solid substrates were recorded using a Varian Cary Eclipse Fluorescence Spectrophotometer. Both the steady-state and time-domain lifetime measurements were carried out using front face illumination. Time-domain lifetime measurements were obtained on a PicoQuant lifetime fluorescence spectrophotometer (Fluotime 100). The excitation source was a pulsed laser diode (PicoQuant PDL800-B) with a 20 MHz repetition rate. The Instrument Response Function (IRF) is about 60 ps. The excitation wavelengths are 285 and 405 nm for 2-AP and 7-HC respectively. Intensity decays were measured through bandpass interference filters. Emission lifetimes were measured with vertically polarized excitation. Magic angle observation was used in the emission path for the time-domain measurements. This optical configuration reduced scattered light of the excitation wavelength without significant distortion of the spectra or lifetimes.

A portion of the sample was cut and mounted on an Al stub with conductive tape, and then observed in an Hitachi SU-70 scanning electron microscope (SEM) directly, i.e., without any further treatment of the sample. Due to the nonconductive substrate (SiO₂), ultra-low voltage was employed for high resolution shallow surface observation and imaging using beam deceleration technology. Samples were surveyed at low magnifications to see the general features and the homogeneity. Representative areas were selected for higher magnification investigation.

FDTD calculations were performed using the program FDTD Solutions (Version 5.0) purchased from Lumerical Solutions, Inc., (Vancouver, Canada). The calculations were performed with the parallel FDTD option on a Dell Precision PWS690 Workstation with the following components: Dual Quad-Core Intel Xeon E5320 processors at 1.86 GHz, and 8 GB RAM. All post-processing of FDTD data were performed using MATLAB (version 7.0) purchased from Mathworks (Natick, Mass.). A time-windowed dipole source, radiating at a fixed wavelength of 375 nm, was used to mimic the emission of 2-AP. This is a soft source, to allow backscattered radiation to pass through it.²⁵ All of the calculations were done assuming a background refractive index of 1.0. The auxiliary differential equations method²⁰ was used to implement a realistic, frequency-dependent and lossy dielectric model for the aluminum nanoparticle. In order to maintain the accuracy and stability of the FDTD calculations, the smallest grid size to accurately model the prescribed system without being computationally prohibitive was obtained in an iterative fashion (convergence testing). In our implementation of FDTD, convergence testing was done by starting the first calculation with a grid size of λ₀/20, where λ₀ is the smallest wavelength expected in the simulation, and then reducing the grid Size by half in sequential simulations and comparing the results of the calculations. Reduction of the grid size was ceased when a grid size (Δ) was approached where results closely match, with the set of results that are obtained from half that particular grid size (Δ/2)²⁵. After testing for convergence, we used a grid size of 2.5 nm for our calculations. The numerical implementation of Maxwell's equations in the FDTD algorithm requires that the time increment Δt have a specific bound relative to the spatial discretization Δ (as mentioned above) to ensure the stability of the time stepping algorithm. In FDTD Solutions, the time step of the simulation is determined by the values of the spatial grid to ensure numerical stability and the user has the flexibility to set the total time of the simulation in femtoseconds (fs).²⁶ Our typical simulations were ranged around 400 fs. This leads to all of our simulations having an excess of 85,000 time steps. Our FDTD software has frequency domain monitors that perform discrete Fourier transforms of the time domain fields while the simulation is running. In this manner, continuous wave (CW) information is obtained at any pre-specified wavelengths for the various field components (Eₓ, E_{y}, E_{z}, Hₓ, H_{y} and H_{z}). Additionally, the time domain monitors can provide time-domain information for the various field components within the FDTD simulation region over the entire course of the simulation. At the end of the simulation, the various field components are checked to see if they decay to zero, thus indicating that the simulation has run for a sufficiently long time for the CW information obtained by Fourier transformations to be valid.²⁶

### Results and Discussion

Surface roughness is known to provide a pathway for the coupling of incident light (plane waves) to surface plasmons and the creation of far-field radiation from plasmons.²⁸ For this reason we used a slow deposition rate to result in a rough metal surface. The surface morphology was controlled principally by the film thickness from 2 to 80 nm. The change of surface morphology with the thickness of Al film was characterized by SEM. A 2-nm film was found to be composed of individual particles. With an increasing thickness of the metal film, it was shown that the particle size became larger. If the Al film was further increased to 40 or 80 nm, the metal film became more and more continuous.

A very thin Al film with thickness of 2 nm did not display a clear plasmon absorbance band in the UV-visible (200-800 nm) spectral range. The absorbance (optical density) of the Al film was increased with increase of thicknesses of Al, however no clear plasmon absorbance bands were observed in any of the evaporated aluminum samples. The size of the particle increased with the thickness of the Al film as observed by SEM images. When the film thickness was over 20 nm, the metal film became continuous without the presence of individual particles.

The emission spectra of spin coated 10 nm PVA film containing 2-AP on 10 nm and 80 nm thick aluminum films mid quartz substrates were determined. We found a significant change in the fluorescence intensity when the fluorophores are in proximity to the particulate Al surface. The emission spectra of 2-AP, which was collected through a 320-nm long-pass filter, showed approximately a 9-fold greater fluorescence intensity from the 10 nm thick particulate aluminum films compared to the control (quartz substrate in the present case). A 4-fold enhanced emission was noticed for 80 nm thick aluminum film compared to quartz control. The enhancement factor is the ratio of the integrated fluorescence intensity of 2-AP in the 325-500 nm spectral region observed on the aluminum nano-structured and control quartz surfaces. This increase in emission intensity of 2-AP is similar to our reports on the MEF phenomenon primarily observed on the silver island films substrates.¹⁰⁻¹² The normalized emission spectra confirmed that the emission spectral properties of the probe, 2-AP, were preserved on both metallic nanostructured and quartz substrates.

The emission spectra of 10 nm PVA film containing 7-HC on quartz, 1.0 nm and 80 nm thick aluminum films were determined. The intensity of 7-HC was increased approximately 6- and 2-fold on 10 nm and 80 nm aluminum films respectively as compared with the control quartz substrate. The normalized emission spectra of 7-HC on quartz and aluminum particulate substrates confirmed that the emission spectral properties of the probe were preserved on both metallic nanostructured and quart substrates. The emission spectral features are similar on both quartz and aluminum substrates except for the difference in intensit, suggesting that the emission spectral properties of 7-HC are maintained on metal nano-structured substrates.

The dependence of fluorescence enhancement factor of PVA film containing 2-AP and 7-HC with the thicknesses of deposited aluminum films was determined. In both of the fluorophores, the highest enhancement was observed when the fluorophores are on 10 nm thick aluminum films. For 2-AP, the enhancement was about only 1.5 fold on 2 nm thick aluminum films. This could be due to the highly particulate nature of the 2 nm thick Al film, there still remains a large area of uncoated quartz, and a major portion of the 2-AP are still on quartz surface. The enhancement factor for 2-AP was consistently around 4-fold for films of 20 nm Al thickness or more. However for 7-HC, the enhancement factor decreased monotonically above 10 nm thick aluminum film.

Without being bound by theory, we believe that this enhanced fluorescence observed from the probes on particulate aluminum films cannot be attributed simply to conventional far-field reflection. Reflection occurs when the ‘metal’ is extended over several wavelengths in size. In the present case, the deposited aluminum particles are smaller than or on the order of the wavelength of light, so these aluminum surfaces essentially behave like particles.

Hence in this case, the fluorophore polarizes the particle and the induced dipole radiates coherently with the fluorophore's dipole. Another important point to note is that in the present case, the fluorophores are approximately within 5 to 1.5 nm away from the metal surface, i.e. essentially in the near-field of metal. In this system, it is thought that the excited state molecule couples to the plasmons on the surface of the aluminum and the combined excited fluorophore-metal complex acts as a unified system, which then radiates. We believe that the observed emission occurs from this near-field metal-fluorophore complex. It is also interesting to note that the extinction spectra of the Al coated slides does not show a distinct plasmon resonance for the Al coated films. However, our fluorescence measurements show consistent enhancements with these films. This is another indication that it is the near-field interaction between the excited state molecule with the surface plasmons of the Al particles that causes the observed enhancement. The plasmon resonances which manifests as distinct peaks in the extinction spectra are essentially far-field quantities that do not occur for the evaporated Al samples, and thus there is no spectral overlap with the absorbance and emission spectra of 2-AP and 7-HC.

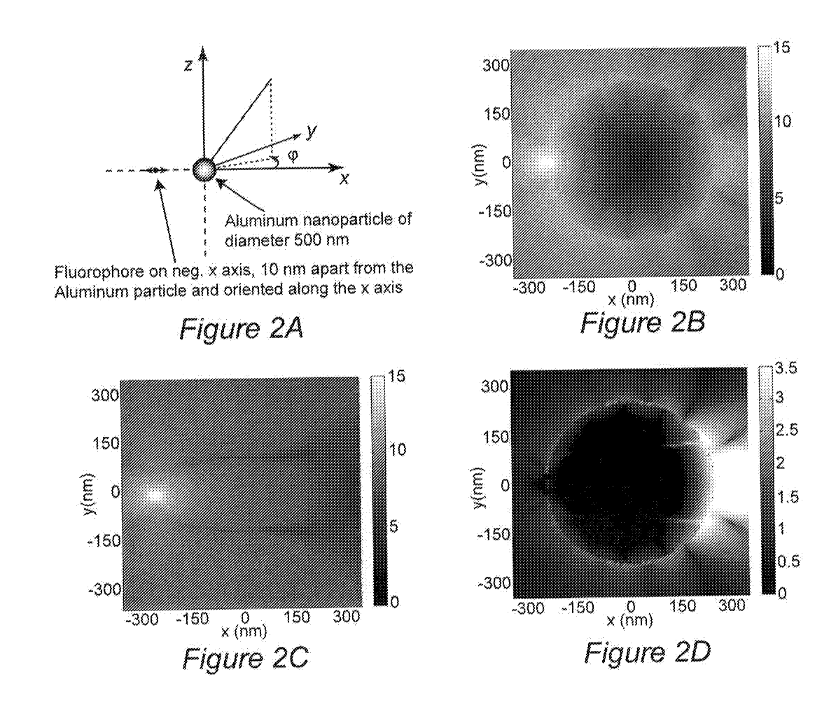

We also examined using FDTD, the electromagnetic near-field distributions around an aluminum particle in the near-field of an excited fluorophore. FIG. 2(a) is a schematic illustration of the system studied. A spherical, aluminum nanoparticle with a diameter of 500 nm is placed at the origin. The SEM images of the 10 nm thick aluminum film (which gave the maximum enhancements) had revealed average particle sizes of approximately 500 nm, and hence we choose this dimension for our calculations. We are aware that the morphology of the actual particles is not exactly spherical, but we choose to use the simplest shape for our calculations. The main objective of the calculations is to investigate whether an excited fluorophore in the near-field of an aluminum nanoparticle can cause field enhancements around the particle. Without being bound by theory, we believe that any near-field enhancements induced by a fluorophore around the aluminum nanoparticle plays a significant role in creating the MEF which we observe experimentally, In our calculations, the fluorophore is placed 10 nm from the surface of the aluminum along the negative x-axis. It is assumed the excitation stage of fluorescence has occurred and the fluorophore is now emitting radiation. We model this radiating fluorophore as an oscillating, point dipole. The fluorophore is oriented with its dipole moment along the x-axis which is normal to the metal surface. FIG. 2(b) shows the electric field intensity in the x-y plane around the 500 nm aluminum nanoparticle separated 10 nm from the fluorophore (oriented along the x-axis). For comparison, FIG. 2(c) shows the intensity around an isolated fluorophore (or oscillating dipole). We have verified that this latter intensity is similar to the near-field of a Hertz dipole.²⁸ FIG. 2(d) is an image of the near-field enhancement that is generated by dividing the intensity around the fluorophore-nanoparticle complex by the intensity around the isolated fluorophore. All the images are displayed in the logarithmic scale (base 10) for clarity of presentation. The scale for FIG. 2(d) has been set from 0 to 1.1 to focus on regions which show enhancement. The areas in FIG. 2(d) which are darkest in color and correspond in the color map to values greater than one are areas where we see maximum enhancements in the near-field around the aluminum particle. It is interesting to observe that the near-field is not enhanced between the particle and the dipole, but is distributed around the nanoparticle with the maximum enhancements on the side of the nanoparticle distal to the fluorophore. Such spatial variations in the near-field enhancements are not easily inferred from experimental observations and thus provide additional insight into the nature of metal enhanced fluorescence.

An important property of metal-enhanced fluorescence is a reduction in lifetime occurring simultaneously with increase in intensity. In fact, shorter lifetimes for fluorophores in close proximity to silver nanostructures, coupled with enhanced emission intensities is indicative of the MEF phenomenon or the radiative decay rate modification, and has been reported in many publications. We have recorded the fluorescence intensity decays of 2-AP and 7-HC on quartz and various thicknesses of aluminum substrates. The intensity-decay data were fit to the multi-exponential model where the intensity decay is given by

$$I ( t ) = {\sum }_{i = 1}^{n} {α}_{i} exp ( - t / {τ}_{i} ) \qquad ( 1 )$$

where αᵢ are amplitude factors associated with each decay time τᵢ. The sum of αᵢ values are normalized to unity, Σᵢαᵢ=1.0. The amplitude-weighted lifetime is given by

$$< τ >= {\sum }_{i} {α}_{i} {τ}_{i} \qquad ( 2 )$$

This value represents the area under the decay curve. The values of αᵢ and τᵢ were determined with the deconvolution of the instrument response function and nonlinear least squares fitting. The goodness-of-fit was determined by the reduced χ₂ value.

The fluorescence intensity decay curves of 2-AP on quartz and 10 nm thick aluminum films were investigated, as was the instrument response function (IRF), Using the best fit to the experimental decay curves, we noted that the intensity decay of 2-AP on aluminum nano-structured surface was faster than observed on quartz control substrate. The amplitude weighted lifetime of 2-AP was found to decrease from 172 ns on the control quartz substrate to 0.45 ns on the 1 nm thick particulate aluminum substrate, a reduction of about 4-fold. This shortening of lifetime on aluminum nano-structured substrate strongly supports that the increase in observed fluorescence intensity is due to the radiation from plasmon-fluorophore complex that results when the aluminum nanoparticles interact with the excited fluorophores in the near-field.

The fluorescence intensity decays of 7-HC on quartz and 10 nm thick aluminum film substrates were also investigated. We observed a faster decay of 7-HC on aluminum surface compared to that on quartz surface. The amplitude weighted lifetime on quartz and 10 nm thick aluminum film is about 1.1 ns and 0.5 ns respectively. These reductions of fluorescence lifetimes are in accordance with our previously reported findings and could be well described by radiating plasmon model^{5,12}. This is because the dyes are within the near-field of the aluminum nanostructures, and couple very efficiently with the particulate aluminum surface, thus inducing radiating plasmons in the aluminum particles. This faster (and hence more efficient) emission from the excited fluorophore-metal complex is the reason behind the observed shortened lifetime, Control measurements on the quartz or aluminum substrates, without the fluorophores (2-AP or 7-HC), yielded almost no signal when observed through the set of bandpass emission filters used to detect the corresponding emission from those fluorophores.

The photophysical properties of a fluorophore is governed by the ratio of magnitude of the radiative decay rate, Γ, with that of the non-radiative decay rates, knr, such as internal conversion and quenching. In general, the emission quantum yield, Φ₀ and lifetime τ₀ of a fluorophore are represented by

Φ₀=Γ/(Γ+κₙᵣ (3)

τ₀=1/(Γ+κₙᵣ (4)

We have found that the enhanced fluorescence intensities or in other words quantum yields of fluorophores in close proximity to metallic nanoparticles and/or nanostructures could be well described by the following equations;

$${τ}_{m} = \frac{1}{Γ + {Γ}_{m} + {κ}_{nr}^{′} } \qquad ( 5 )$$

where Υₘ and κₙᵣ′ are radiative and nonradiative rates in presence of metal particles respectively. The lifetime of a fluorophore in presence of metal is decreased by an increased (system) radiative decay rate as. By system, we mean the excited state fluorophore-metal complex:

$${Φ}_{m} = \frac{Γ + {Γ}_{m}}{Γ + {Γ}_{m} + {κ}_{nr}^{′}} \qquad ( 6 )$$

It is apparent from the above equations that in presence of metal particles or surfaces as radiative decay rate Υm increases, the quantum yield Φₘ increases, while the emission lifetime, τₘ, decreases.

The dependence of the ratio of amplitude weighted lifetimes of 2-AP on the thickness of aluminum fi ms was determined. The largest reduction of emission lifetime was observed when the probe 2-AP was on the 10 nm thick aluminum film. As mentioned earlier, we have also observed the largest enhancement in the emission intensity of both the fluorophores 2-AP and 7-HC on 10 nm thick particulate aluminum substrates. Without being bound by theory, we believe the shorter lifetimes of both probes observed on aluminum nano-structured surface are due to an increase in the radiative decay rate, but we cannot rule out quenching of some portion of the population. The amplitude-weighted lifetime displayed a tendency of increase with the thickness of the aluminum film above 10 nm.

In many examples of fluorescence based sensing, it is fluorophore detectability that governs the utility and sensitivity of the sensing approach. In general, the detectability of a fluorophore is determined by two factors: the extent of background emission from the sample and the photostability of the fluorophore. Accordingly, we have made a comparison between the photostability, of the two fluorophores on glass and Al Substrates.

The photostability of both the fluorophores (2-AP and 7-HC) on quartz and particulate aluminum film substrates was determined. Using the same incident excitation power, we observed significantly more fluorescence from the Al nano-structured substrates as compared to the quartz control sample (data not shown). The incident excitation power on the Al nanostructured films was attenuated to give a similar initial emission intensity (at time=0 min) as observed on the quartz substrate. The results showed that both probes (2-AP and 7-HC) are more photostable on the 10 nm thick particulate aluminum substrate as compared to the quartz substrate.

It is interesting to comment on the total detectability of fluorophores in presence of the metal nano-structures/particles. While a maximum of 8-9 fold increase in fluorescence intensity is clearly beneficial, a reduced fluorescence lifetime of probes also enables the system to be cycled faster, as the lifetime of a species determines its cyclic rate. Hence, 8-fold increase in intensity coupled with a 4-fold reduction n in lifetime of the probes in proximity to the aluminum nano-structured surfaces provides a significant increase in detectability. In addition, a reduced lifetime also affords for increased fluorophore photostability as there is less time for excited state photodestructive processes to occur. In total, it appears that the detectability of UV-blue fluorophores can be increased significantly near aluminum particles considering the co-operative effects of enhanced fluorescence intensity, reduced lifetime and increased photostability on particulate aluminum substrates.

### SUMMARY

in this Example, we have introduced particulate aluminum films as substrates for MEF in the ultraviolet-blue spectral region. We investigated the variation in the size of the aluminum nanostructures to identify the optimum conditions for MEF in the ultra-violet-blue spectral region. We have observed a maximum of ˜9-fold increase in emission intensity and a 4-fold reduction in fluorescence lifetime from a DNA base analogue 2-aminopurine (2-AP) in proximity to the aluminum nano-structured surfaces that provides increased detectability when compared to the quartz control. A maximum of ˜6-fold increased in fluorescence intensity and about 3-fold decreased in lifetime was attained for the probe 7-HC on particulate aluminum films. We have also observed increased photostability of the both fluorophores on aluminum substrates. Our results indicate that the observed emission arises due to the near-field metal-fluorophore complex. The observation of MEF an particulate aluminum films suggests the use of these stable nanostructures in surface-localized bioassays. These robust particulate aluminum substrates could be useful in many fluorescence based assay applications such as DNA arrays as these substrates are very stable in the buffers containing chloride salts compared to usual silver nanoparticle based substrates for ME E UV fluorophores are routinely used to label proteins and membranes. Examples include diphenylhexatriene and its derivatives, pyrenes, dansyl, and anilinonaphthalene-type fluorophores. Based on the results using 2-AP, such fluorophores should also display MEF. The optical properties of aluminum could potentially also be useful for obtaining MEF with intrinsic protein fluorescene. Medical assays and biotechnology applications such as drug discovery frequently utilize surface-localized chemistry. Our results on MEF with particulate aluminum surfaces indicate that the range of wavelengths can be extended down to the ultraviolet. This will provide a wide range of wavelengths for development of multi-analyte assays using different color fluorophores.

### REFERENCES FOR EXAMPLE 1

(1) van Dyke, K. Luminescence immunoassay and molecular applications; CRC Press: Boca Raton, 1990.
(2) Hemmila, A. Application of fluorescence in immunoassays; John Wiley & Sons: New York, 1992.
(3) Walker, N. J. Science 2002, 296, 557-559.
(4) Livak, K. J.; Flood, S. A.; Marmaro, J.; Giusti, W.; Deetz, K. PCR Methods Appl. 1995, 4, 357-362.
(5) Lakowicz, J. R. Anal. Biochem 2001, 298, 1-24.
(6) Sokolov, K.; Chumanov, G.; Cotton, T. M. Anal. Chem. 1998, 70, 3898-3905,
(7) Chumanov, G.; Sokolov, K.; Gregory, B. W.; Cotton, T. M. J. Phys. Chem. 1995, 99, 9466-9471.
(8) Geddes, C. D.; Cao, H.; Gryczynski, I.; Gryczynski, Z.; Fang, J. Y.; Lakowicz, JR. J. Phys. Chem. A 2003, 107, 3443-3449.
(9) Messinger, B. J.; von Raben, K. U.; Chang, R. K.; Barber, P. W. Phys. Rev. B 1981, 24, 649-657.
(10) Ray, K.; Badugu, R; Lakowicz, J. R. Am. Chem. Soc. 2006, 128, 8998-8999.
(11) Ray, K.; Badugu, R; Lakowicz, J. R. Langmuir 2006, 22, 8374-8378.
(12) Lakowicz, J. R. Anal. Biochem. 2005, 337, 171-194,
(13) Novotny, L.; Hecht, B. Principles of Nano Optics, Cambridge University Press: Cambridge, 2006.
(14) Yguerabide J.; Yguerabide, E. E. Anal. Biochem. 1998, 262, 137-156,
(15) Yguerabide J.; Yguerabide, E. E. Anal. Biochem 1998, 262, 157-176
(16) Anger, P.; Bharadwaj, P.; Novotny, L. Phys. Rev. Lett. 2006, 96, 113002.
(17) Kühn, S.; Häkanson, U.; Rogobete, L.; Sandoghdar, V. Phys. Rev. Lett. 2006, 97, 017402,
(18) Weitz, D. A.; Garoff S.; Gersten. J. I.; Nitzan, A. J. Chem. Phys 1983, 78, 5324-5338.
(19) Gryczynski, I.; Malicka, J; Gryczynski, Z.; Lakowicz, J. R. Anal. Chem. 2004, 76, 4076-4081.
(20) Taflove A, Hagness, S. C. Computational Electrodynamics: The Finite-Difference Time-Domain Method; Artech House: Boston, 2000.
(21) Sullivan, D. M. Electromagnetic simulation using the FDTD method, IEEE Press: New York, 2000.
(22) Yang P.; Liou, N. K. J. Opt, Soc. Am. A 1996, 13, 2072-2085.
(23) Gray. S. K., Kupka, T. Phy. Rev. B. 2003, 68, 045415.
(24) Chang, S. H.; Gray, S. K.; Schatz, G. C. Optics Express 2005, 13, 3150-3165.
(25) Tatlove, A.; Brodwin, M. E. IEEE Trans. Microwave Theory and Techniques 1975, 23, 623-630.
(2.6) Reference Guide for FDTD Solutions™ Release 5.0, 2007, http://www.lumerical.com/fdtd.
(27) Raether, H. Surface Plasmons on Smooth and Rough Surfaces and on Gratings; Springer-Verlag: New York, 1998, pp. 136.
(28) Shadowitz, A. The Electromagnetic Field; Dover: New York, 1988.
(29) Lakowicz, J, R. Principles of Fluorescence Spectroscopy; 3rd Edition; Springer: New York, 2006.

### Example 2

### On the Possibility of Using Aluminum Nanoparticles as Substrates for Metal-Enhanced Fluorescence in the Ultra-Violet Region for the Label Free Detection of Biological Molecules

We used the finite-difference time-domain method to predict that aluminum nanoparticles can be used as efficient substrates for metal-enhanced fluorescence in the ultra-violet region for label free detection of biological molecules, Our calculations focus on the fluorophore emission in the range of 300-420 nm which is typical of intrinsic fluorescence emission of proteins. In all of our calculations, the fluorophore is modeled as a radiating point dipole with orientation defined by its polarization. When a fluorophore is oriented perpendicular to the metal surface, our calculations reveal a large increase in total power radiated through a closed surface containing the fluorophore-metal nanoparticle system, in comparison to the isolated fluorophore. In contrast, significant emission quenching occurs if the fluorophores are oriented parallel to the metal surfaces. Our results also indicate that the radiated power enhancement of a system containing a broadband dipole source placed in proximity to various sized aluminum nanoparticles when compared to identical silver nanoparticles consistently show higher enhancements in the ultra-violet region, thus suggesting that aluminum is a better substrate for fluorescence enhancement in this region. We also show that the radiated power enhancement for fluorophores next to aluminum nanoparticles is strongly dependent on the nanoparticle size, fluorophore-nanoparticle spacing and fluorophore orientation. An increase in radiated power indicates increases in the relative radiative decay rates and decreases in relative lifetimes of the emission near the nanoparticles. Additionally we see that the radiated power enhancement is dramatically increased when the fluorophores are located between two aluminum nanoparticles of a dimer system. We also examine the effect of the incident excitation and the excited fluorophore on the near-field around the nanoparticles.

The extinction, scattering and absorption cross section of a 20 nm Al nanoparticle was determined. The extinction spectra is a sum of the absorption and scattering spectra and is dominated by the absorption. The plasmon resonance manifested as peaks at approximately 150 nm. The extinction, scattering and absorption efficiency of a 20 nm Al nanoparticle was determined. The extinction efficiency is a sum of the absorption and scattering and is dominated by the absorption. The plasmon resonance manifested as peaks at approximately 150 nm. And important observation is that for a 20 nm Al nanoparticle, the extinction coefficient revealed that the extinction cross sections of these particles are several times larger than the physical cross section.

Radiated power enhancement for a dipole spaced 5 and 10 nm respectively from a 20 nm Al nanoparticle was determined, Dipoles radiating at different wavelengths from 100-700 nm (in 1 nm intervals) were used to compute the spectra. All the dipoles in this calculation were oriented perpendicular (along x-axis) to the surface of the Al nanoparticle.

Note that Lumerical's FDTD software self normalizes the power output of an isolated dipole to 1, so any value of power greater than 1 represents a n enhancement. The normalized extinction spectra of a 20 nm Al nanoparticle were determined. The normalization of the radiated power enhancement was done with respect to the peak at ˜163 nm, and the normalization of the extinction spectra was done with respect to the peak at ˜160 nm. The results showed that the radiated power enhancement spectra overlaps with the extinction spectra of 20 nm Al. All the dipoles in this calculation were oriented perpendicular to the surface of the Al nanoparticle.

The extinction, scattering and absorption cross section of a 40 nm Al nanoparticle were determined. The extinction, spectra is a sum of the absorption and scattering spectra, and the extinction is dominated by the scattering. The plasmon resonances manifest as dipolar peaks at approximately 180 nm. We also noted higher order modes as shoulders at approximately 130 nm. The extinction, scattering and absorption efficiency of a 40 nm Al nanoparticle was also determined. The extinction efficiency is a sum of the absorption and scattering and the extinction is dominated by the scattering. The plasmon resonances manifested as dipolar peaks at approximately 180 nm. We also saw higher order modes as shoulders at approximately 130 nm. An important observation is that for a 40 nm Al nanoparticle, the extinction efficiency reveals that the extinction cross sections of these particles are several times larger than the physical cross section.

Radiated power enhancement for a dipole spaced 5 and 10 nm respectively from a 40 nm Al nanoparticle was also determined. Dipoles radiating at different wavelengths from 100-700 nm (in. 1 nm intervals) were used to compute the spectra, All the dipoles in the calculation were oriented perpendicular to the surface of the Al nanoparticle. The normalized extinction spectra of a 40 nm Al nanoparticle were determined. The normalization of the radiated power enhancement was done with respect to the most red-shifted peak at ˜180 nm, and the normalization of the extinction spectra was done with respect to the dipole peak at ˜170 nm. The results showed that the radiated power enhancement spectra overlaps with the extinction spectra of 40 nm Al. All the dipoles in this calculation were oriented perpendicular to the surface of the Al nanoparticle.

The extinction, scattering and absorption cross section of an 80 nm Al nanoparticle were also determined. The extinction spectra is a sum of the absorption and scattering spectra and the extinction is dominated by the scattering. The plasmon resonances manifested as dipolar peaks at approximately 250 nm. We also noted higher order modes at approximately 170 nm n and 140 nm. The extinction, scattering and absorption efficiency of an 80 nm Al nanoparticle were also determined. The extinction efficiency is a sum of the absorption and scattering, and the extinction is dominated by scattering. The plasmon resonances manifested as dipolar peaks at approximately 250 nm. We also saw higher order modes at approximately 170 nm and 140 nm. An important observation is that for an 80 nm Al nanoparticle, the extinction efficiency reveals that the extinction cross sections of these particles are several times larger than the physical cross section.

The radiated power enhancement for a dipole spaced 5 and 10 nm respectively from Al an 80 nm Al nanoparticle were also determined. Dipoles radiating at different wavelengths from 100-700 nm (in 1 nm intervals) were used to compute the spectra. All the dipoles in this calculation were oriented perpendicular to the surface of the Al nanoparticle. The normalized radiated power enhancement for a dipole spaced 5 and 10 nm respectively from an 80 nm. Al nanoparticle, and the normalized extinction spectra of an 80 nm Al nanoparticle, were determined. The normalization of the radiated power enhancement was done with respect to the most red-shifted peak at ˜330 nm, and the normalization of the extinction spectra was done with respect to the dipole peak at ˜260 nm. The results showed that the radiated power enhancement spectra overlaps with the extinction spectra of 80 nm Al. All the dipoles in this calculation were oriented perpendicular to the surface of the Al nanoparticle.

The radiated power enhancement for a dipole spaced 5 and 10 nm respectively from an 80 nm Al nanoparticle was determined. Dipoles radiating at different wavelengths from 100-700 nm (in 1 nm intervals) were used to compute the spectra. All the dipoles in this calculation were oriented parallel (along y-axis) to the surface of the Al nanoparticle, and an enhancement below 1.0 represented quenching. Normalized radiated power enhancement for a dipole spaced 5 and 10 nm respectively from an 80 nm Al nanoparticle, and the normalized extinction spectra of an 80 nm Al nanoparticle, were determined. The normalization of the radiated power enhancement was done with respect to the most red-shifted peak at ˜150 nm, and the normalization of the extinction spectra was done with respect to the dipole peak at ˜260 nm. The results showed that the radiated power enhancement spectra overlaps with the extinction spectra of 80 nm Al. All the dipoles in this calculation were oriented parallel to the surface of the Al nanoparticle.

The extinction, scattering and absorption cross section of a 100 nm Al nanoparticle were determined. The extinction spectra is a sum of the absorption and scattering spectra and is dominated by the scattering. The plasmon resonances manifest as dipolar peaks at approximately 300 nm. Higher order modes at approximately 190 nm, 160 nm and 140 nm were also observed. The extinction, scattering and absorption efficiency of a 100 nm Al nanoparticle were also determined. The extinction efficiency is a stun of the absorption and scattering, and also is dominated by scattering. The plasmon resonances manifested as dipolar peaks at approximately 300 nm. Higher order modes at approximately 190 nm, 160 nm and 140 nm were also observed. An important observation is that for 100 nm Al nanoparticles, the extinction efficiency revealed that the extinction cross sections of these particles are several times larger than the physical cross section.

The radiated power enhancement for a dipole spaced 5 and 10 nm n respectively from a 100 nm Al nanoparticle was determined. Dipoles radiating at different wavelengths from 100-700 nm (in 1 nm intervals) were used to compute the spectra. All the dipoles in this calculation were oriented perpendicular to the surface of the Al nanoparticle. The normalized radiated power enhancement for a dipole spaced 5 and 10 nm respectively from a 100 nm Al nanoparticle, and the normalized extinction spectra of a 100 nm Al nanoparticle were determined. The normalization of the radiated power enhancement was done with respect to the most red-shifted peak at ˜400 nm, and the normalization of the extinction spectra was done with respect to the dipole peak at ˜310 nm. The results showed that the radiated power enhancement spectra overlaps with the extinction spectra of 100 nm Al. All the dipoles in this calculation were oriented perpendicular to the surface of the Al nanoparticle.

The radiated power enhancement for a dipole spaced 5 and 10 nm respectively from a 100 nm Al nanoparticle was determined, Dipoles radiating at different wavelengths from 100-700 nm (in 1 nm intervals) were used to compute the spectra. All the dipoles in this calculation were oriented parallel to the surface of the Al nanoparticle, and an enhancement below 1.0 represented quenching. The normalized radiated power enhancement for a dipole spaced 5 and 10 nm respectively from a 100 nm Al nanoparticle, and the normalized extinction spectra of an 80 amu Al nanoparticle, were determined. The normalization of the radiated power enhancement was done with respect to the most red-shifted peak at ˜160 nm, and the normalization of the extinction spectra was done with respect to the dipole peak at ˜310 nm. The results showed that the radiated power enhancement spectra overlaps with the extinction spectra of 100 nm Al. All the dipoles in this calculation were oriented parallel to the surface of the Al nanoparticle.

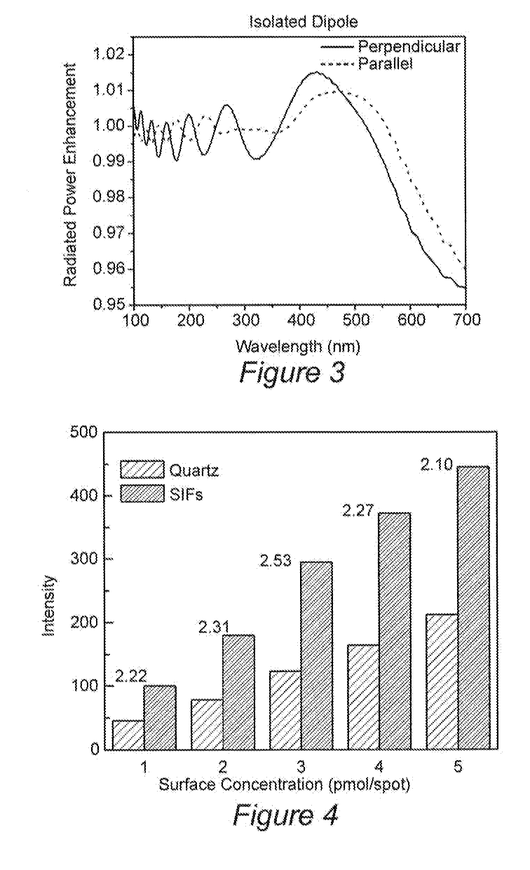

FIG. 3 depicts the result of control calculations showing that the radiated power enhancement for an isolated dipole oriented both in the perpendicular direction and parallel direction is always approx. 1 for all the dipole wavelengths used between 100-700 nm.

### Example 3

### Metal-Enhanced Fluorescence of Intrinsic Protein Tryptophan Residues

### Application to Label-Free Bioassays.

### Abstract

The detection of submonolayers of proteins based on native fluorescence is a potentially valuable approach for label-free detection. We have examined the possibility of using silver nanostructures to increase the emission of tryptophan residues in proteins. Fluorescence spectra, intensities, and lifetimes of multi-layers and sub monolayers of proteins deposited on the surface of silver island films were measured. Increased fluorescence intensities from two to three-fold and similar decrease in lifetimes were observed in the presence of the silver nanoparticles compared to the proteins on the surface of the bare quartz. The observed spectral effects of silver particles on tryptophan fluorescence indicates the possibility for the design of analytical tools for the detection of proteins without traditional labeling,

### Introduction

Fluorescence detection is a central technology in clinical chemistry, drug discovery, proteomics, genonmics, and cell biology. Almost without exception, detection is accomplished using extrinsic fluorophores which are used to label the biomolecules. The need for labeling of the biomolecules with extrinsic fluorophores results in increased costs and complexity. Because of this added complexity, there is a rapidly growing interest in methods which provide label-free detection. A wide variety of surface-based methods are being tested for label-fee detection of binding. These methods include surface plasmon resonance (SPR) [1-2], surface-enhanced Raman scattering [3-4], electrochemical approaches [5-6], nanowires [7], optical microcavities [8], optical transmission [9-10], reflectivity [11], interferometry [12], and photonic crystals [13]. The most widely used label-free detection method is SPR, which is being extended to high throughput capabilities by the use of imaging modality [14-16]. The sensing mechanism of SPR is based on high sensitivity of reflected light at specific surface plasmon resonance angle. The system uses a thin continuous metal film (˜40-50 nm) of gold or silver to which molecules bind or dissociate causing detectable changes in the refractive index in the interface between metallic film and bulk solution. The quantitative interpretation of SPR signal to adsorbed biomolecules and estimated detection sensitivity of about 1-10 pg/mm2 are described elsewhere [17-18]. Another approaches for construction of label-free detection were exploited the localized surface plasmon resonance (LSPR) of metallic nanostructures.

Transmission surface plasmon resonance (T-SPR) was introduced for measurements changes in extinction peak of plasmon spectra of discontinuous gold films in the visible an near-infrared wavelength range. [19] Wavelength shift of extinction spectra of metallic nanoparticles on dielectric substrates and nanoholes in gold film have also been demonstrated for optical biosensors [20-21].

In this report we describe the use of intrinsic protein fluorescence for general detection of binding reactions on surfaces. Proteins possess three intrinsic fluorophores, phenylalanine (Phe), tyrosine (Tyr), and tryptophan (Trp). The emission from proteins is dominated by tryptophan because of its longer excitation and emission wavelengths, good quantum yield, and fluorescence resonance energy transfer from tyrosine to the tryptophan residues (FRET) [22]. On average, tryptophan is present at 1.3% of the amino acid residues which results in many proteins containing multiple tryptophan residues. For example, a typical protein with molecular weight 50 kDa will contain about 400 amino acid residues, and thus an average 5.2 tryptophan residues. The abundance of tryptophan residues in proteins is both an advantage and disadvantage for fluorescence label-free detection. The abundance is an advantage because most target proteins will contain this intrinsic label and a disadvantage because all the other proteins in the sample will also contain tryptophan and be fluorescent. For this reason tryptophan fluorescence has not been used in analytical applications. However, there are several recent reports on using UV fluorescence for detection and quantification of protein interactions [23-27]. Relatively high detection sensitivities were reported using frequency tripled output of Ti:sapphire lasers, 1-5 ng per spot on two-dimensional gel electrophoresis for several proteins [23], and better than 10 pM of antibody BP53-12 bound to immobilized p53 on nitrocellulose membrane [26].

We believe that recent progress in metal-enhanced fluorescence (MEF) can be used for high sensitivity detection of target proteins in samples which contain other tryptophan-containing proteins. The MEF approach is based on short range interactions of fluorophores with metallic nanostructures which, depending on the metal geometry, occur at distances from 5 to 100 nm. The fluorophores can be excited by the evanescent fields due to surface plasmons, rather than by propagating light. The emission can be modified by the presence of a nearby metal structured surface. The metal structure can result in more rapid emission of the fluorophore, or may change the normally isotropic emission into directional emission. These spectral changes are due to near-field non-radiative interactions of the fluorophores with the metal surfaces. For fluorophores, which absorb and emit at visible wavelengths, it has been shown in many experiments that proximity to silver and/or gold particles results in increases of intensity, radiative decay rate, and photostability [28-29]. The observations of MEF due to silver and aluminum nanostructures have been also reported for the UV wavelength range [30-33].

### Materials and Methods

Preparation of The silver Island films and Surface Deposition of Proteins

The wet chemical deposition method was used to coat the quartz substrate with the silver island films (SIFs). The procedure of deposition of SIFs on glass and quartz substrates has been described elsewhere [29, 34]. The wet chemical deposition technique results in a variability in the particle sizes and shapes as has been previously shown using atomic force microscopy with particle sizes up to 500 nm and thicknesses of 50-100 nm [29]. The absorption spectrum maximum of the fabricated SIFs is near 450 nm with optical density of about 1.15, which indicate that the particles were of sub-wavelength size. We used high density silver islands to limit the void areas between particles where the proteins can also adsorb and tryptophan residues would not effectively interact with particle plasmon resonance.

The protein used were biotinylated bovine serum albumin (BSA-Bt) from Sigma Aldrich (St. Louis, Mo.), avidin from Pierce Biotechnology (Rockford, Ill.) and immunoglobulins (IgG₃ and anti-IgG₃) from Southern Biotech (Birmingham, Ala.). For deposition of proteins on the SIFs and bare quartz substrates, we used direct deposition of proteins by spotting from aqueous solution and by non-covalent electrostatical immobilization. Because the fluorescence of tryptophan molecule is relatively weak in comparison with organic fluorophores, mostly due to its low extinction coefficient (5,500 M⁻¹cm⁻¹ at 280 nm) and UV spectral range where autofluorescence can be usually high, we performed initial experiments using avidin. This protein contains 16 tryptophan residues and is relatively bright compared to other proteins. First, avidin was deposited through spotting a water solution on the SIFs surface that allowed an increase in protein concentration on the surface and facilitated easy measurements of tryptophan fluorescence. Secondly, a layer-by-layer method was used to deposit layers of biotinylated bovine serum albumin (BSA-bt) and avidin. The initial BSA-Bt layer was electrostatically immobilized on the SIFs and quartz through incubation of solution of 20 ug/ml in phosphate buffer at pH 7.4 for 1 hour. After washing out unbound BSA-Bt, the avidin solution of 10 ug/ml was incubated for 1 hour creating one layer of BSA-Bt-Avidin. The procedure was repeated to construct up to four BSA-Bt-Avidin layers. The adsorbed layer of BSA-Bt on the substrate with SIFs and various concentrations of avidin were also used to demonstrate the model protein assay.

In addition, a monolayer of anti-IgG was immobilized on the SIFs substrate followed with a blocking solution and binding of IgG₃ was performed.

### Spectroscopic Measurements

The extinction spectra of silver island films were measured using a single beam spectrophotometer (Hewlett-Packard model 8543). The emission spectra of avidin were measured using Varian fluorometer (Eclipse 4) with excitation of 280 nm.

Fluorescence lifetimes were measured using frequency-domain fluorometer (K2 from ISS, Champaign, Ill.). The excitation source was frequency-doubled output from rhodamine 60 dye laser pumped with mode-locked argon ion laser. The excitation was 280 nm and emission observed through band, pass filter was 320-360 nm. We are aware that the tryptophan fluorescence properties, quantum yield, and lifetime strongly depend on the environment [35], thus experiments were performed at identical conditions for samples on the bare quartz and on the SIFs.

The intensity decays were fit to the multi-exponential model

$$I ( t ) = {\sum }_{i = 1}^{n} {α}_{i} exp ( - t / {τ}_{i} ) \qquad ( 1 )$$

where τᵢ is the decay time and the αᵢ is the amplitude

$$( {\sum }_{i} α = 1.0 ) .$$

Intensity (τ_{M}) and amplitude (τ>) weighted lifetimes were calculated for comparison between values on bare quartz and on SIFs

$$< τ >= {\sum }_{i} {α}_{i} {τ}_{i} , {τ}_{M} = \frac{fi τ i }{{\sum }_{i} fi τ i} \qquad ( 2 )$$

where the fractional intensity is defined as

$${f}_{i} = \frac{{α}_{i} {τ}_{i}}{{\sum }_{i} {α}_{i} τ i} \qquad ( 3 )$$

### Results and Discussion

### Avidin Layers Spotted on the SIFs

FIG. 4 shows the intensities of avidin on the quartz and SIFs surfaces with consecutive spotting. Water solution of avidin with 1 uM concentration was spotted in 1 uL aliquots (1 pmol of avidin) creating a spot of about 5 mm diameter. Assuming that avidin is uniformly distributed within the spot area, one can estimate the maximal surface density to be about 5.1 pmol/cm² which corresponds approximately to a monolayer of avidin. The calculated surface density of a full crystalline monolayer of streptavidin (similar size to avidin) is about 2.8 ng/mm2 [36], which corresponds to about 4.4 pmol/cm². The intensity measurements were performed by illuminating the whole spot to facilitate comparison of signals from an equal number of avidin molecules spotted on quartz and SIFs. The whole spot was illuminated with excitation light at 280 nm and emission spectra were recorded. Sequentially added 1 uL sample volumes resulted in increased surface density of avidin and in proportionally increased intensities. The intensities on the SIFs were more than 2-fold larger than an the quartz. The experiment with spotted avidin demonstrates that detection of less than 66 ng (1 μl of 1 μM) avidin is possible which is better than 1 μg per protein band in detecteion with polyacrylamide gels using UV absorbance and native protein fluorescence [24-25]. The optimization of metallic nanostructures will lead to higher enhancement factor than 2-fold in the present experiment and likely the sensitivity to be achieved in the range of 1-10 ng per spot as shown for more sophisticated system that uses high power IV laser for excitation and CCD camera [23].

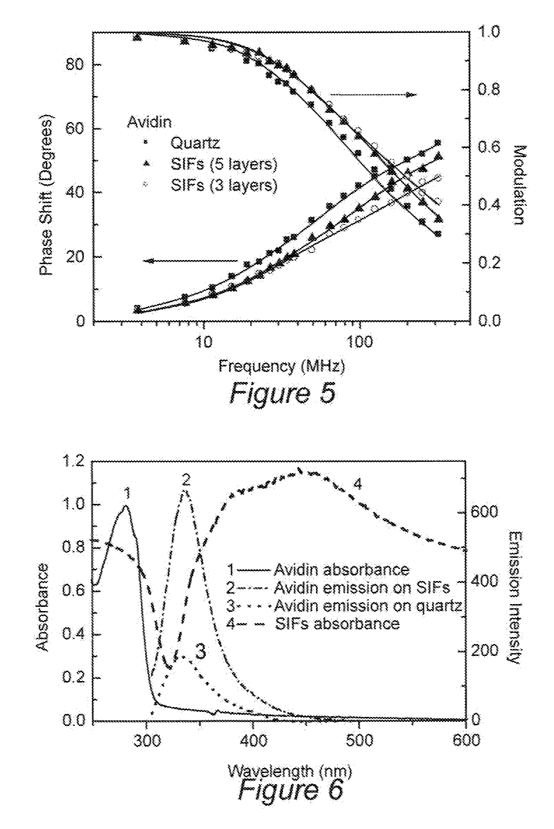

The lifetime measurements for avidin spotted on the quartz and SIFs with three and five 1 uL aliquots are shown in FIG. 5. The detailed analysis of intensity decays are summarized in Table I. The amplitude weighted lifetime decreased 1.8-fold on SIFs compared to the quartz for three microliters (approximately 3 avidin layers) and only 1.08-fold for five microliters (about S avidin layers). The average thickness of single avidin layer is comparable to that for streptavidin which was determined as bout 5 nm [36]. The simultaneous increase in intensity and decrease in lifetime is attributed to an increase of radiative decay rate of fluorophore due to interaction with metal particle plasmons.

TABLE I
Analysis of intensity decays of the avidin deposited on quartz
slide and on SIFs. Excitation wavelength at 280 nm (frequency
doubled output of R6G dye laser), emission observed through
band pass filter 360/40 nm.

| Avidin | τᵢ (ns) | αᵢ | fᵢ | τ > (ns) | τ_{M} (ns) |
|---|---|---|---|---|---|
| On quartz | 5.50 | 0.064 | 0.313 | 1.13 | 2.72 |
| | 1.89 | 0.294 | 0.493 | | |
| | 0.34 | 0.642 | 0.194 | | |
| On SIFs | 3.73 | 0.070 | 0.419 | 0.63 | 1.94 |
| ~3 layers | 0.95 | 0.227 | 0.346 | | |
| | 0.21 | 0.703 | 0.235 | | |
| On SIFs | 4.17 | 0.064 | 0.341 | 1.05 | 2.04 |
| ~3 layers | 1.23 | 0.299 | 0.472 | | |
| | 0.23 | 0.637 | 0.188 | | |

Photobleaching of avidin deposited on quartz and on SIFS was similar resulting in about 50% loss of initial intensities after continuous illumination (over 20 minutes) of focused excitation light at 280 nm.

The observed increase in intensities and decrease in lifetime of avidin of about 2-fold due to the presence of SIFs is somewhat unexpected in view of recent theoretical calculations of effects of particle plasmons on radiative and non-radiative decay rates of fluorophores with emission in UV wavelength range [37]. The theoretical calculations considered single silver particles, while our observations are for plurality of particles of various shapes and sizes that construct the SIFs, usually with broad plasmon spectrum. Additionally, closed spaced particles can interact with each other. The plasmon resonance spectrum of SIFs relative to the absorption and emission spectra of the avidin is shown in FIG. 6. The absorption spectrum of avidin is in aqueous solution and normalized (to 1.0) to the maximum at 280 nm. The emission spectra of avidin are on the SIFs and quartz substrates for three layers (BSA-Br-Avidin). It is interesting that the minimum in the extinction spectrum of SIFs (minimum of imaginary part of dielectric function of silver) overlaps partially with the emission spectrum of tryptophan. At this time we do not have a clear explanation for what the effect of the “plasmon dip” has on the enhancement of the tryptophan fluorescence. Nonetheless, the observed effects of simultaneous increase in intensity, decrease in lifetime, and increase in photostability (photobleaching rate same as on quartz but about 2-fold higher intensity) suggest that tryptophan residues interact with silver particle plasmons similarly as has been observed for fluorophores in visible and NIR wavelength range [29]. This indicates that the silver nanostructures can be utilized for enhanced tryptophan fluorescence and applied for label-free detection schemes where sub mono layer of proteins on the surface can be detected. To obtain larger intensity enhancements of tryptophan residues, a further optimization of the silver nanostructures is needed. Fabrication of silver nanoparticles with smaller sizes will result in shift of the plasmon resonance peak to shorter wavelength and will likely result in larger effects on the emission properties of the tryptophan.

Variation of Protein Thickness using Layer-by-Layer Protein Immobilization

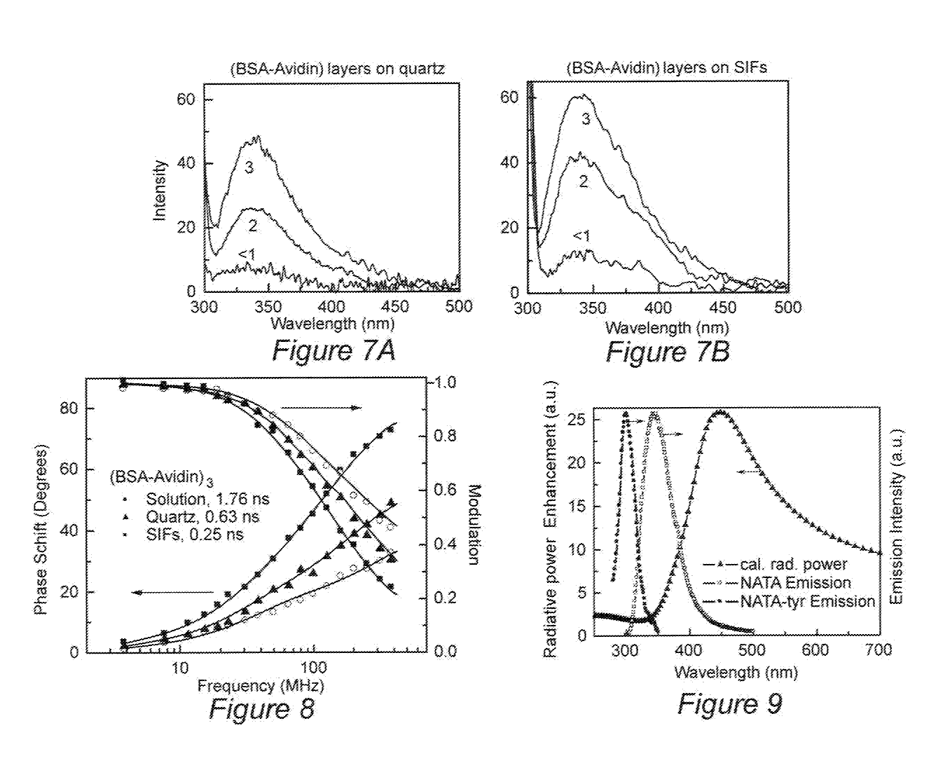

The initial experiments with simple spotting deposition illustrated that multi-layers of protein can be easily detected on the quartz and SIFs. Due to the spotting deposition and drying of the sample, the environments for tryptophan residues are not the same as for proteins in biological samples. In the next step of experiments, we constructed subsequent monolayers of BSA-Bt and avidin in PBS buffer pH 7.4 [38] and measured relative intensities and lifetimes on the (BSA-Bt-Avidin)n multi-layers, where n=1, 2, 3, and 4. The multi-layer system also provided the means to investigate the distance effect of SIFs on fluorescence of tryptophan residues. The protein multi layer system does not provide a single distance because the tryptophan residues are distributed throughout the protein, Nonetheless, the obtained data have a value for future design of label-free assays. FIG. 7A-B shows the emission spectra of the two and three layers of (BSA-Bt-Avidin) constructed on quartz (A) and on SIFs (B).

For lifetime measurements and relative intensities a band pass filter (320-360 nm) was used. The observed lifetimes on SIFs were shorter than on the quartz for all multi-layer proteins while the intensities were larger. This observation is consistent with those observed for avidin spotted on surfaces. A detailed frequency-domain intensity decays of avidin in solution and for three layers of (BSA-Bt-Avidin) on quartz and on SIFs are shown in FIG. 8. It is also observed that the lifetime of avidin on quartz is shorter than for avidin in buffer solution. This is due to environmental difference to which tryptophan is sensitive [35]. Similar effect of decreased fluorescence lifetime of tryptophan residues have been observed for immobilized p53 peptide on nitrocellulose and subsequently for bound BP53-12 antibodies [26].

### Biotinylated Bovine Serum Albumin and Avidin as a Model Label-Free Assay

The experiment involving spotted avidin and measurements of its intensity and lifetime indicate that detection of sub monolayers of protein on SIFs surfaces is possible. To illustrate the usefulness of this observation, we performed a model label-free bioassay using a SIF surface onto which the layer of biotinylated BSA was electrostatically deposited. Subsequently, the SIFs substrates were exposed to the avidin solutions at concentrations ranging from 1 ng/ml to 10 ug/ml (15 pM to 154 nM). The surface biotin concentration is determined by the 9 biotin molecules per BSA which allows that avidin molecule can bind to 2 BSA biotin sites [39], thus the 1:1 avidin-to-BSA binding can be assumed. The intensity measurements were performed in buffer after washing away unbound avidin. The binding of avidin to the surface immobilized biotinylated BSA was measured. Titration data were fit to the Langmuir adsorption isotherm:

$$θ = \frac{K [ A ]}{1 + K [ A ]} \qquad ( 4 )$$

where the θ is the faction of the bound avidin of the total number of available binding sites on the surface (θ˜I_{A}/I_{A}^{max}), where I_{A} is intensity at avidin concentration [A] and I_{A}^{ma} is intensity at saturation), K is the surface binding constant and [A] is the avidin concentration. The fit to the Eq, (4) resulted in surface binding constant of 9×10¹¹ M⁻¹. This binding constant is smaller than expected in solution between avidin and biotin of 10¹³-10¹⁵M⁻¹, however, a similar lower value was also observed for silver surface binding constant between streptavidin and biotin [20].

Upon binding of avidin to the surface, the intensity increased 7.7-fold which is more than the ration of equimolar concentrations of BSA-Bt and avidin in the buffer solution of 3.5-fold. The higher intensity ratio of avidin to BSA found on SIFs is likely due to potential quenching of emission of BSA because the average tryptophan residues distance is less than 3 nm from silver islands, while the tryptophans in avidin is separated by larger distance where the silver quenching effects are expected much less. The large increase in intensity is due to a large number of tryptophan residues in the avidin molecule relative to those in BSA more optimal distance from the SIFs.

One can estimate the amount of avidin bound to the surface that can be detected assuming that maximal avidin surface concentration is similar to that of the BSA-Bt saturation surface coverage, typically in the range from 1.20-180 ng/cm² (1.8-2.7 pmol/cm²) [40-41]. Titration data showed that with our proof of principle system, we are capable of 3 detecting the 1-5% of maximal avidin surface concentration. This indicates that less than 2 ng/cm²²of avidin generated the measurable signal on top of a full monolayer of BSA. One can forecast that optimization of metallic nanostructures and assays will result in the detection capabilities using intrinsic protein fluorescence comparable to the SPR systems in the range of 100 pg/cm^{2 [}17-18]. One should note that detection sensitivity for SPR systems are dependent on molecular weight while detection sensitivity of proteins based on UV fluorescence is dependent on the tryptophan density of the binding biomolecule. As a result it will be possible to design capture proteins which do not contain tryptophan residues, and one can use unlabeled aptamers which do not absorb and emit at the wavelengths used for tryptophan.

Label Free Potential with Blocking Solution

When designing a label free detection method, consideration has to be given to the potential signal from a blocking agent used in most surface-based assays. The blocking solution usually contains proteins that adhere well to most dielectric and metallic surfaces and minimizes or prevents non-specific binding, We used IgG₃ as a capture antibody, a protein based blocker formulation used in Western blotting and ELISA applications (StartingBlock (PBS) Blocking Buffer from Pierce Biotechnology) and anti-IgG₃. The blocking solution displayed strong fluorescence with a peak intensity of about 340 nm which was comparable to about 20 uM of IgG₃ in solution. However, despite its strong fluorescence, the intensity from the surface with immobilized capture IgG₃ increased only 64% and 80%, after blocking solution on quartz and SIFs, respectively. This increased intensity due to the blocking solution can be regarded as moderate, and in future assays, the intensity due to blocking reagents can be even less if covalent immobilization of capture antibodies will be per formed with higher surface density or using blocking solutions without proteins.

Contacting the quartz and SIFs surfaces with anti-IgG₃ caused marked increase in intensities that demonstrates the potential of detection of binding biomolecules to the surface in the presence of blocking protein agents. In contrast to SPR where the signal is proportional to the mass density of bound protein (g/mm¹), the detection based on intrinsic protein fluorescence is proportional to the surface density of tryptophan (mol/mm²). This means label-free fluorescence-based detection will be favorable for small proteins with tryptophan residues compared to the SPR detection

The important question is how the bulk concentration of the same analyte or combined signal from biomolecules in the sample will affect the signal from the bound analyte to the MIFF surface?The most abundant protein in biological samples is human serum albumin (HAS) which display similar tryptophan fluorescence as BSA, Assuming conservatively the surface density of capture antibody as I pmol/cm² (150 ng/cm) (antibody size approximately 10 nm×10 nm) and the bulk solution of SA of 1 mg/ml, one can find that the ratio of capture antibodies to the free HSA in solution thickness of 1 μm is 15:1.

Assuming further that the intensity of bound analyte will be enhanced by MEF within the distance of 5-30 nm one can realize that label-free detection using protein fluorescence can be accomplished in the presence of bulk sample solution without washing steps.

### Conclusion

We have demonstrated a potential of using silver nanostructures for enhancement of tryptophan fluorescence, Four experiments with various configurations of surface bound proteins provided promising results for design of label-free detection based on intrinsic protein florescence. The intensity and lifetime measurements confirmed that the fluorescence of tryptophan residues is enhanced due to tryptophan-particle plasmon interactions. The fluorescence signal from a sub monolayer of avidin of less than 2 ng/cm2 has been detected.

### REFERENCES FOR EXAMPLE 3

1. X. Yu D. Xu, Q Cheng. Label-free detection methods for protein microarrays. Proteomics 6 (2006) 5493-5503.
2. M. A, Cooper, Non-optical screening platforms: the next wave in label-free screening?Drug Discov. Today 11 (2006) 1068-1074.
3. L. Sun. C. Yu, J. Irudayaraj. Surface-enhanced raman scattering based nonfluorescent probe for multiplex DNA detection. Anal, Chem, 79 (2007) 3981-3988.
4. A. E. Grow, L. L. Wood, J. L. Chaycomb, P. A. Thompson. New biochip technology for label-free detection of pathogens and their toxins. J. Microbiol, Methods 53 (2003) 221-233.
5. M. Vestergaard, K. Kennan, E. Tamiya. An overview of label-free electrochemical protein sensors. Sensors 7 (2007) 3442-3458.
6. J. A. Hansen, J. Wang, A-N, Kawde, Y. Xiang, K. V. Gothelf, G. Collins (2006) Quantum-dot/aptamer-based ultrasensitive multi-analyte electrochemical biosensor. J. Am. Chem. Soc. 128 (2006) 2228-2229.
7. F. Patolsky, G. Zheng, C. M. Lieber, Fabrication of silicon nanowire devices for ultrasensitive, label-free real-time detection of biological and chemical species. Nature Protocols 1(4) (2006) 1711-1724.
8. A. M. Armani, R. P, Kulkarni, S. E. Fraser, R. C. Flagan, K. J. Vahala. Label-free, single molecule detection with optical microcavities. Science 317 (2007) 783-786.
9. G, Acharya, C-L. Chang, C. Savran. An optical biosensor for rapid and label-free detection of cells. J. Am. Chem. Soc., 128 (2006) 3862-3863,
10. B. J, Zeskind, C. D. Jordan, W. Timp, L Trapani, G. Waller, V. Horodincu, D. J. Ehrlich, P. Matsudaira, Nucleic acid and protein mass mapping by live-cell deep-ultraviolet microscopy, Nature Methods 4(7) (2007) 567-569.
11. J. P. Landry, X. D. Zhu, J. P Gregg, Label-free detection of microarrays of biomolecules by oblique-incidence reflectivity difference microscopy. Optics Letts. 29(6) (2004) 581-xxx,
12. M. M. Varma, H. D. Inerowicz, F. Regnier, D. D. Nolte. High-speed label-free detection by spinning-disk. Biosensors and Bioelectronics 19 (2004) 1371-1376,
13, P. C. Mathias, N. Ganesh L. L. Chan, B. T, Cunningham. Combined enhanced fluorescence and label-free biomolecular detection with a photonic crystal surface. Applied Optics 46(12) (2007) 2351-2360,
14. K. S. Phillips. Q. Cheng. Recent advances in surface plasmon resonance based techniques for bioanalysis. Anal. Bioanal. Chem. 387 (2007) 1831-1840.
15. G. J. Wegner, A. W. Wark, H. J. Lee, B. Codner, T. Sacki, S, Fang, R. M. Corn. Real-time surface plasmon resonance imaging measurements for the multiplexed determination of protein adsorption/desorption kinetics and surface enzymatic reactions on peptide microarrays. Anal. Chem. 76 (2004) 5677-5684.
16. H. J. Lee, D. Nedelkov, R. M. Corn (2006), Surface plasmon resonance imaging measurements of antibody arrays for the multiplexed detection of low molecular weight protein biomarkers. Anal. Chem. 78(18) (2006) 6504-6510.
17. L. S. Jung, C. T. Campbell, T. M. Chinowsky, M. N. Mar, S. S. Yee, Quantitative Interpretation of the Response of Surface Plasmon Reonance Sensors to Adsorbed Films, Langmuir 14 (1998) 5636-5648.
18. J. Homola, S. S. Yee, G. Gauglitz, Surface plasmon resonance sensors: review. Sensors and Acuators B 54 (1999) 3-15.
19. M. Lahav, A. Vaskevich, I. Rubinstein, Bological Sensing Using Transmission Surface Plasmon Resonance Spectroscopy, Langmuir 20 (2004) 7365-7367.
20. A. J. Haes, S. R. P, Van Duyne. A Nanoscale Optical Biosensor: Sensitivity and selectivity of an Approach Based on the Localized Surface Plasmon Resonance Spectroscopy of Triangular Silver Nanoparticles. J. Am. Chem. Soc. 124 (2002)
10596-10604.
21. J. C. Ribobh, A. J. Haes, A. D. McFarland, C. R. Yonzon, R. P. Van Duyne, A Nanoscale Optical Biosensor: Real-Time Immunoassay in Physiological Buffer Enabled by Improved Nanoparticle Adhesion, J. Phys., Chem, B 107 (2003) 1772-1780.
22. J. R. Lakowicz. Principles of Fluorescence Spectroscopy, 3rd Edition, Springer, New York, 2006, pp. 954.
23. J. Roegener, P. Lutter, R. Reinhardt, M. Blüggel, H. E. Meyer, D. Anselmetti. Ultrasensitive Detection of Unstained Proteins in Acrylamide Gels by Native UV Fluorescence. Anal. Chem. 75 (2003) 157-159.
24. C. Sluszny, E. S. Yeung. One- and Two-Dimensional Miniaturized Electrophoresis of Proteins with Native Fluorescence Detection. Anal. Chem. 76 (2004) 1359-1365.
25. P. Schulze, M. Ludwig, F. Kohler, D. Belder, Deep UV Laser-Induced Fluorescence Detection of Unlabeled Drugs and Proteins in Microchip Electrophoresis. Anal. Chem. 77 (2005) 1325-1329.
26. M. Schüttpeltz, C. Müller, H. Neuweiler, M. Saner. UV Fluorescence Lifetime Imaging Microscopy: A Label-Free Method for Detection and Quantification of Protein Interactions. Anal. Chem. 78 (2006) 663-669.
27. H. A. Engström, P. O. Andersson, S. Ohlson. A label-free continuous total-internal-reflection-fluorescence-based immunosensor. Anal. Biochem. 357 (2006) 159-166.
28. J. R. Lakowicz. Radiative decay engineering: Biophysical and biomedical applications. Anal. Biochem. 298 (2001) 1-24.
29. J. R. Lakowicz, Y. Shen, S. D'Auria, J. Malicka, J. Fang, Gryczynski, L. Gryczynski. Radiative decay engineering, 2. Effects of silver island films on fluorescence intensity, lifetimes, and resonance energy transfer. Anal, Biochem. 301 (2002) 261-277.
30. J. R. Lakowicz, B. Shen, Z. Gryczynski, S. D'Auria, 1, Gryczynski, Intrinsic Fluorescence from DINA Can Be Enhanced by Metallic Particles. Biochem. Biophys. Res. Commun. 286 (2001) 875-879.
31. I, Gryczynski, J. Malicka, J. Lukomska, Z. Grczynski, J. R. Lakowicz. Surface Plasmon-coupled Polarized Emission of N-Acetyl-L-Tryptophanamide. Photochem. Photabiol. 80 (2004) 482-485.
32. Gryezynski, J. Malicka, Z. Gryczynski, K. Nowaczyk, J. R. Lakowicz, Ultraviolet Surface Plasmon-Coupled Emission Using Thin Aluminum Films. Anal. Chem. 76 (2004)
4076-4081.
33, K. Ray, M. H. Chowdhury J. R. Lakowicz, J. R. Aluminum Nanostructured Films as Substrates for Enhanced Fluorescence in the Ultraviolet-Blue Spectral Region. Anal. Chem. 79 (2007) 6480-6487.
34. F. Ni, T. M, Cotton, Chemical procedure for preparing surface-enhanced Raman scattering active silver films, Anal. Chem. 58 (1986) 3159-3163.
35. D. Toptygin, R. S. Savtchenko, N. D. Meadow, S. Roseman, I. Brand. Effect of the Solvent Refractive Index on the Excited-State Lifetime of a Single Tryptophan Residue in a Protein, J. Phys, Chem. B 106 (2002) 3724-3734.
36. S. A. Darst, M. Ahlers, P. H. Meller, E. W. Kubalek, R. Blakenburg, H. O, Rib, H. Ringsdorf; R. D. Kornberg. Two-dimensional crystals of streptavidin on biotinylated lipid layers and their interactions with biotinylated macromolecules. Biophys. J., 59 (1991) 387-396.
37, H. Mertens, A. F. Koendrink, A. Polman, Plasmon-enhanced luminescence near noble-metal nanospheres: Comparison of exact theory and an improved Gersten and Nitzan model. Phys. Rev, B 76 (2007) 115123.
38. T. Hoshi, H. Saiki, J. Anzai. Layer-by-layer deposition of avidin and biotin labeled antibody on a solid surface to prepare a multilayer array of antibody. J. Chem. Soc., Perkin Trans. 2 (1999) 1293-1294.
39. L. S. Jung, K. E. Nelson, P. S. Stayton, C. T. Campbell, Binding and Dissociation Kinetics of Wild-Type and Mutant Streptavidins on Mixed Biotin-Containing Alkylthiolate Mono layers. Langmuir 16 (2000) 9421-9432,
40. V. Silin, H. Weetall, D. J. Vanderah, SPR Studies of Nonspecific Adsorption Kinetics of Human IgG and BSA on Gold Surfaces Modified by Self-Assembled. Monolayers (SAMs). Colloid Interface Sci. 185 (1997) 94-103.
41. C-G. Gölander, E. Kiss, protein adsorption on functionalized and ESCA-characterized polymer films studied by ellipsometry. J. Colloid Interface Sci. 121 (1998) 240-253.

### Example 4

### Metal-Enhanced Intrinsic Fluorescence of Proteins on Silver Nanostructured Surfaces towards Label-Free Detection

In recent years metal-enhanced fluorescence (MEF) using silver particles has been reported for a number of fluorophore emitting at visible wavelengths. However it was generally thought that silver particles would always quench fluorescence at shorter wavelengths. We now report the observation of metal-enhanced fluorescence of the tryptophan analogue N-acetyl-L-tryptophanmnide (NATA) on silver nano-structured surfaces. NATA is a model for the intrinsic tryptophan emission from proteins. We have also studied the effects of silver nanostructures on the emission of N-acetyl-L-tyrosinamide (NATA-tyr). In the case of NATA we observed increased emission, decrease in fluorescence lifetimes, and increase in photostability when NATA was embedded in 15 nm thick spin-casted poly(vinyl alcohol) film on silver nanostructured surfaces. We have also investigated the effects of silver nanostructures on the emission from thin poly(vilnyl alcohol) films containing NATA-tyr. However, we have observed, no increase in fluorescence signal for NATA-tyr on silver nanostructures. To understand these results we performed numerical calculations using the Finite-Difference Time-Domain (FDTD) technique to model a tryptophan-wavelength dipole near a spherical silver particle. Our calculations reveal an enhancement of the power of the radiated emission by the excited-state fluorophore in proximity to a 100 nm silver nanoparticle covering the emission spectra of NATA and NATA-tyr. These calculations show a clear wavelength dependence with the specific spectral region displaying low-enhancement at the shorter NATA-tyr wavelength and higher enhancement at NATA emission wavelength. Our FDTD calculations also reveal that excited fluorophores in the near-field of a 100 nm silver nanoparticle can induce enhancement fields of varying degrees of the intensity of the near-fields around the particle that is dependent on the wavelength of the emission. Without being bound by theory, we believe this enhanced near-fields play a role in our observation of MEF from metal surfaces. The enhanced emission of NATA on silver nanostructures suggests that the extension of MEF to the UV region opens new possibilities t study tryptophan-containing proteins without labeling with longer wavelength fluorophores towards label free detection of biomolecules.

### Introduction

Fluorescence detection presently is a central technology in the biosciences. The applications of fluorescence include cell imaging, medical diagnostics and biophysical research. Another growing use of fluorescence is for measurements of a large number of samples as occur on. DNA arrays, protein, arrays and high throughput screening (HTS). HTS typically includes testing of a large number of small molecules for biological activity, most often drug-receptor interactions. Almost all the applications of fluorescence require the use of labeled drugs and labeled biomolecules, which becomes increasingly inconvenient as the number of compounds to be tested has increased. The need for labeling of the biomolecules with extrinsic fluorophores results in increased costs and complexity. Because of this added complexity there is a rapidly growing interest in methods which provide label-free detection (LFD), as has been described in recent reviews.¹⁻³ Perhaps the most widely used LFD method is surface plasmon resonance (SPR). SPR detection is based on measurements of small change in refractive index of a sample on a thin gold film.⁴⁻⁵ These changes are detectable because the SPR angle is sensitive to the change in refractive index which occurs when target molecules bind to capture molecules on the gold surface. Because of its general applicability SPR is being extended to high throughput capabilities by the use of SPR imaging.⁶⁻⁸ The importance of label-free detection can be seen from the large number of other methods which are being tested for label-free detection.

Proteins possess three intrinsic fluorophores, phenyalanine (phe), tyrosine (try) and tryptophan (trp). Proteins are highly fluorescent, which is due primarily to tryptophan residues because of its longer excitation and emission wavelengths, good quantum yield, and fluorescence resonance energy transfer (FRET).⁹ Tryptophan residues in proteins occur with a frequency of 1.3% of the amino acid residues and are thus present in almost all proteins. Tryptophan-free proteins are relatively rare. However, since most proteins contain tryptophan, this emission may not be specific for proteins of interest in a biological sample. Additionally, proteins are excited near 280 nm and emit near 350 nm. These conditions result in high background fluorescence from most samples. For these reasons intrinsic tryptophan emission from proteins might not be used to detect specific proteins. In this paper, we use metallic nanostructures to enhance the fluorescence of the tryptophan analogue N-acetyl-L-tryptophanamide (NATA) near silver nanostructures. NATA contains amide residues in the amino and carboxyl sides of third-carbon and is thus an analogue of tryptophan in proteins. We also studied the tyrosine analogue N-acetyl-L-tyrosinamide (NATA-tyr). The most important fact is that we observe the enhancement of tryptophan fluorescence when NATA is close to the metal nanostructured surfaces. This allows design of surface-based assays with biorecognitive layer that specifically bind the protein of interest and thus enhance its intrinsic fluorescence. Large increases in fluorescence intensity and decreases in lifetime provide the means of direct detection of bound protein without separation from the unbound.

We also used numerical calculations in the form of the Finite-Difference Time-Domain (FDTD) technique to understand our experimental results. FDTD is an implementation of Maxwell's time-dependent curl equations for solving the temporal variation of electromagnetic waves within a finite space that contains a target of arbitrary shape, and has recently become the state-of-the-art method for solving Maxwell's equations for complex geometries.¹⁰⁻¹⁸ A major advantage of FDTD is that it is a direct time and space solution, and hence offers the user a unique insight into a myriad of problems in photonics. More information on the FDTD technique can be found in Refs. 10-18. Our calculations reveal that the enhancement of the power of the radiated emission of an excited-state fluorophore in proximity to a 100 nm silver nanoparticle shows clear wavelength dependence with specific spectral regions displaying high emission enhancements while other regions showing only modest or no enhancements. Our FDTD calculations also reveal that fluorophores in the near-field of a 100 nm silver nanoparticle can induce enhancements of varying degrees of the intensity of the near-fields around the particle that is dependent on the wavelength of the emission. Without being bound by theory, we believe these enhanced fields play a role in our observation of MEF from metal surfaces.

### Experimental

Silver island films (SIFs) on quartz slides were prepared as described previously.¹⁹⁻²¹ NATA and low molecular weight polyvinyl alcohol (PVA, MW 13000˜23000) were purchased from Aldrich chemical company and used as received, N-acetyl-L-tyrosinamide (NATA-tyr) was obtained from Acros Organics. NATA and NATA-tyr in solutions of 0.5 wt % polyvinyl alcohol (PVA) was spin coated onto quartz and SIFs slides.

Absorption spectra were collected using a Hewlett-Packard 8453 spectrophotometer. SIFs displayed the characteristic surface plasmon resonance with an absorption maximum near 450 nm. Fluorescence spectra of probes on solid substrates were recorded using a Varian Cary Eclipse fluorescence spectrophotometer. Both the steady-state and time-domain lifetime measurements were carried out using front face illumination. Time-domain lifetime measurements were obtained on a Pico-Quant lifetime fluorescence spectrophotometer (Fluotime 100).

The fluorescence intensity decays were analyzed in terms of the multi-exponential model⁹:

$$I ( t ) = {\sum }_{i = 1}^{n} {α}_{i} exp ( - t / {τ}_{i} ) \qquad ( 1 )$$

In this expression τᵢ are the decay times and αᵢ are the amplitudes and

$${\sum }_{i} α = 1.0 .$$

The fractional contribution of each component to the steady-state intensity is described by:

$$fi = \frac{{α}_{i} {τ}_{i}}{{\sum }_{j} α j τ j} \qquad ( 2 )$$

The average lifetime is represented by:

$$\overline{τ} = {\sum }_{i} fi τ i \qquad ( 3 )$$

and the amplitude-weighted lifetime is given by:

$$< τ >= {\sum }_{i} α i τ i \qquad ( 4 )$$

The values of αᵢ and τᵢ were determined using the PicoQuant Fluofit 4.1 software with the deconvolution of instrument response function and no linear least squares fitting. The goodness-of-fit was determined by the reduced χ² value.

A portion of the SIFs sample was cut and coated with a thin layer of gold (approx. 5 nm) in a sputter coating system. This step was done to minimizes charging effects during scanning electron microscope (SEM) imaging. The sample was then mounted on an Al stub with conductive tape, and observed in a Hitachi SU-70 SEM. Due to the nonconductive substrate (glass), low voltage (3 kV) was employed for high resolution shallow surface observation and imaging using beam deceleration technology. Samples were surveyed at low magnifications to see the general features and the homogeneity. Representative areas were selected for higher magnification investigation. SEM image of the SIFs surface showed the nanoscale heterogeneity of the silver particles' sizes, shapes and spatial distributions. From this SEM image, we observed the average size of these silver particles is about 100 μm. FDTD Calculations: 3-dimensional FDTD calculations were performed using the program FDTD Solutions (Version 5.0) purchased from Lumerical Solutions, Inc., (Vancouver, Canada). In all of our calculations, it is assumed that the excitation stage of fluorescence has occurred and the fluorophore is now emitting dipole radiation. A time-windowed dipole source, radiating at a fixed wavelength of 350 nm, was used to mimic the emission of NATA. Similarly, we used a dipole source radiating at 305 nm to mimic the emission of NATA-tyr. This is a soft source, to allow backscattered radiation to pass through it. In order to maintain the accuracy and stability of the FDTD calculations, the smallest spatial grid size to accurately model the prescribed system without being computationally prohibitive was obtained in an iterative fashion. This process is called convergence testing. In our implementation of FDTD, convergence testing was done by starting the first calculation with a grid size of λ₀/20, where λ₀ is the smallest wavelength expected in the simulation, and then reducing the grid size by half in sequential simulations and comparing the results of the calculations. The reduction of the grid size was stopped when we approached a grid size (Δ) where results closely match with the set of results that are obtained from half of that particular grid size (Δ/2), and that is also computationally feasible.^{10-11, 16-18} For our calculations, we employed a grid size of 2 nm. The numerical implementation of Maxwell's equations in the FDTD algorithm requires that the time increment Δt have a specific bound relative to the spatial, discretization Δ (as mentioned above) to ensure the stability of the time stepping algorithm.^{10-11,16-18} Typically the durations of our simulations were 400 fs, corresponding to an excess of 200,000 time propagation steps for each calculation. The FDTD package employed has frequency domain monitors that perform discrete Fourier transforms of the time domain fields while the simulation is running in this manner, continuous wave (CW) information is obtained at any pre-specified wavelengths for the various electric and magnetic field components. All of the calculations were done assuming a background relative dielectric constant of 1.0.

The enhancement in the total radiated power is inferred by integrating the normal flux passing through a closed surface containing the system is given by:

P_{rad}/P₀ (4)

where a system is either an isolated dipole (excited-state fluorophore) or a fluorophore in proximity to a silver nanoparticle, P₀ is the radiated power of a classical dipole in a homogeneous background which in our case is air/vacuum, and P_{rad} is the radiated power of the dipole in proximity to the silver nanoparticle. In our calculations, since we use a radiating dipole source to model the excited fluorophore, the power from an isolated dipole P₀ is used for normalization.^{18,22-23} Note that P_{rad}/P₀>1 represents enhancement and P_{rad}/P₀<1 represents quenching. We used a set of six frequency domain surface monitors to create a box around the system, and measured the total power radiated by the system by integrating the real part of the Poynting vector over all six surfaces. The power was normalized to the analytic expression for the power radiated by a dipole in a homogeneous dielectric (in this case, air/vacuum) to get the relative change in power radiated as described in Eq. 4.

### Results and Discussion

Emission spectra of NATA spin coated from PVA solution for quartz and silver nanostructured substrates were determined. The emission spectral distribution of NATA measured on metallic nanostructures and quartz is essentially identical and characteristic for tryptophan. The SIFs gave an enhancement of approx. 8-fold when compared to the quartz. The normalized spectra had a high degree of overlap thus suggesting that there was no change in the spectral properties of the NATA when it interacts with the silver nanostructures.

The lifetimes of 15-nm thick PVA film containing NATA on quartz and SIF substrates were investigated. The intensity decay of NATA on SIFs surface was faster than observed on the quartz control substrate. The intensity-decay of NATA PVA film on quartz could be fitted with a single exponential with a lifetime of 3.2 ns. NATA on SIFs surface could only be fitted with a double-exponential with two lifetimes of 132 nsec (19%) and 0.98 nsec (81%). The amplitude-weighted lifetime of NATA on SIFs was 1.4 nsec. Hence, the intensity decays showed that the lifetime was decreased to about 2.5-fold. In the case of NATA on the SIF it is likely that the more complex multi-exponential decay reflects the presence of NATA molecules close to and more distant from the silver surface. This shortening of lifetime on the silver nanostructured substrate supports the notion that the increase in observed fluorescence intensity is due to the radiation from the plasmon-fluorophore complex^{24,25} that results when excited fluorophores interact with silver particles in the near-field. The reduction of lifetime of NATA on SIFs also suggests an increase in the radiative decay rate of NATA due to the silver particles.²⁶⁻²⁸ We note that precise agreement between the increases in intensity and decrease in lifetime is not expected.

The lifetime was estimated from a single exponential model. It is well known that time-domain measurements often result in over-weighting of the lifetime by the longer lifetime components in heterogeneous decay, particularly when the decay of the short components overlaps the instrument response function.

The emission spectra of spin coated 15-nm PVA film containing NATA-tyr on SIFs and quartz substrates was investigated. The emission spectra of NATA-tyr collected through a 300-nm long-pass filter showed similar fluorescence intensities for both SIFs and quartz substrates. The emission spectrum of NATA-tyr is slightly blue-shifted on SIFs compared to quartz, We observed a slightly faster decay for the NATA-tyr PVA film on SIFs compared to that on quartz. Control measurements on the quartz or SIFs surfaces, without NATA or NATA-tyr, yielded almost no signal when observed through the set of band-pass emission filters used to detect the corresponding emission from those probes.

In general, the detectability of a fluorophore is determined by two factors: the extent of background emission from the sample and the photostability of the fluorophore. We examined the effects of silver island films on the photostability of NATA. The photostability of NATA on quartz and SIFS substrates was determined. Using the same incident excitation power, we observed significantly more fluorescence from the silver nanostructured substrates as compared to the quartz control sample (data not shown). When the incident excitation power on the SIFs was attenuated to give the similar initial emission intensity as observed on the quartz substrate, it was evident from that NATA is more photostable on the SIFs substrate as compared to the quartz substrate. This result is consistent with an increase in the radiative decay rate of NATA in presence of silver nanoparticles, and also with the decreased lifetimes of NATA on SIFs. While a maximum of 8-fold increase in fluorescence intensity of NATA is clearly beneficial, a reduced fluorescence lifetime of probes also enables the system to be cycled faster, as the lifetime of a species determines its cyclic rate. Hence, 8-fold increase in intensity coupled with a 2.5-fold reduction in lifetime of the NATA in proximity to the silver nanostructured surfaces provides a significant increase in detectability. In total, it appears that the detectability of NATA can be increased significantly near silver nano-particles considering the co-operative effects of enhanced fluorescence intensity, reduced lifetime and increased photostability on silver nanostructured surface.

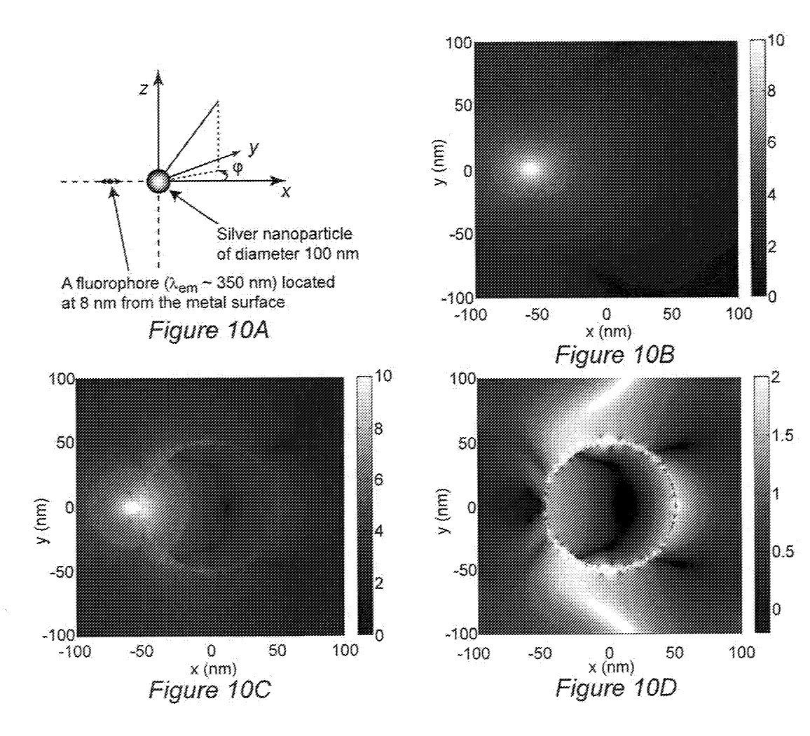

It was surprising to observe metal-enhanced fluorescence at these UV wavelengths. Hence we questioned whether the observed effects were consistent with the known optical properties of silver. We used the FDTD method to calculate the power radiation by a dipole near silver particles, and the wavelength dependence of the effects. FIG. 9 shows the radiated power enhancement for a dipole spaced 8 nm from a 100 nm Ag nanoparticle. The SEM images of the SiFs revealed average particle sizes of approximately 100 nm diameter, and hence we choose this dimension for our calculations. We are aware that the morphology of the actual particles in SIFs is not exactly spherical, but we choose to use the simplest shape for our calculations. In FIG. 9, dipoles radiating at different wavelengths from 250-700 nm (in 1 nm intervals) were used to compute the spectra. All the dipoles in this calculation were oriented perpendicular to the surface of the Ag nanoparticle. Our FDTD calculations self normalizes the radiated power output from a system comprising the Ag nanoparticle and a dipole to the output of an isolated dipole, so any value of radiated power greater than 1 represents an enhancement. The output of an isolated dipole is always 1. It is interesting to note that the normalized radiated power increases rapidly at 350 nm. Also shown in the figure are the emission spectra of NATA and NATA-tyr on SIFs. This figure shows that the NATA emission has a much larger spectral overlap with the radiated power enhancement spectra than the NATA-tyr spectra. This may be a reason why NATA shows good enhancements with SiFs while NATA-tyr does not.

We have also used FDTD to calculate the enhancements in the intensity of the near-fields around the Ag nanoparticles that is induced by the fluorophore. FIG. 10A is a schematic illustration of the system studied. A spherical, silver nanoparticle with a diameter of 100 nm is placed at the origin. The main objective of the calculations is to investigate whether an excited fluorophore in the near-field of a silver nanoparticle can cause field enhancements around the particle at the UV wavelength region. Without being bound by theory, we believe that any near-field enhancements induced by a fluorophore around the silver nanoparticle plays a significant role in creating the MEF which that we observe experimentally. The fluorophore is oriented with its dipole moment along the x-axis which is normal to the metal surface and placed 8 nm from the surface of the Ag nanoparticle. FIG. 10B shows the intensity around an isolated fluorophore (or oscillating dipole). We have verified, as might be expected, that this latter intensity is similar to the near-field of a Hertz dipole.²⁹ We have chosen the wavelength off the dipole as 350 nm to match the emission maximum of the NATA. FIG. 10C shows the electric field intensity in the x-y plane around the 100 nm silver nanoparticle separated 8 nm from the fluorophore (oriented along the x-axis). FIG. 0D is an image of the near-field enhancement that is generated by dividing the intensity around the fluorophore-nanoparticle complex by the intensity around the isolated fluorophore (i.e. dividing FIG. 10C by FIG. 10B). All the images are displayed in the logarithmic scale (base 10) for clarity of presentation. The areas in FIG. 10D which are green, yellow and red in color correspond in the color map to values greater than one are areas where we see strong enhancements in the near-field around the silver particle. It is interesting to observe that the near-field is not enhanced between the particle and the dipole, but is distributed in an interesting “wing” shaped pattern around the nanoparticle with the maximum enhancements on the on the central area of this “wing” shape. We also observe appreciable near-field enhancements in the side of the Ag nanoparticle opposite to the fluorophore. Such spatial variations in the near-field enhancements are not easily inferred from experimental observations and thus provide additional insight into the nature of metal enhanced fluorescence.

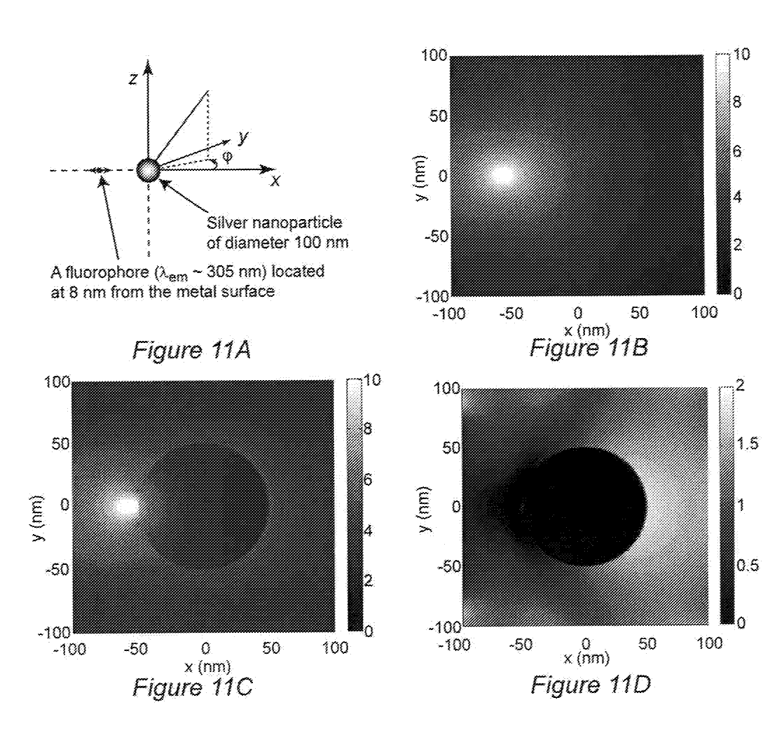

FIG. 11 also presents the near-field intensity distributions of a fluorophore-silver nanoparticle system. However in this case, the wavelength of the dipole chosen is 305 nm which corresponds to the emission maxima of NATA-tyr. The images of FIG. 11 were generated in an identical fashion to FIG. 10. FIG. 11D is an image of the near-field enhancement that is generated by dividing the intensity around the 305 nm dipole-Ag nanoparticle complex by the intensity around the isolated 305 nm dipole (i.e. dividing FIG. 11C by FIG. 11B. FIG. 11D shows the enhancement in the near-fields induced around the Ag nanoparticle by the 305 nm emitting dipole is quite modest when, compared to the 350 nm emitting dipole (FIG. 11D). We again see that the slight near-field enhancements observed are not between the particle and the dipole, but occurs on the side of the Ag nanoparticle opposite to the dipole. Hence a direct comparison of FIG. 10 and FIG. 11 tells us that the near-field intensity plots agrees qualitatively with our experimental observations where we see a significant enhancement of the NATA emission but not the NATA-tyr emission with SiFs.

### Conclusions

The presence of silver nano-particles significantly increases the brightness of the intrinsic fluorescence of NATA. The lifetimes of the NATA are shorter on silver particles than on quartz substrate. On the other hand, we have not observed any significant effect of silver nanoparticles on the emission of NATA-tyr. We have presented a numerical FDTD study of the effect on the emission of excited fluorophores near a silver nanoparticle and contrasted our results with an isolated fluorophore. In these numerical calculations, we focus only on the emission side of fluorescence, Inspection of intensity patterns reveals how, in the near field, very specific regions around the nanoparticles experience field enhancements and quenching. This type of result is not easily inferred from far-field observations and is relevant to potential applications that would involve spatially resolved molecular spectroscopy or detection using fluorescence. The extension of MEF to the UV region opens new possibilities to study tryptophan-containing proteins without labeling with longer wavelength fluorophores and provides an approach to label-free detection of biomolecules.

### REFERENCES FOR EXAMPLE 4

1. Ramachandran, N.; Larson, D. N.; Stark, P. R. H.; Hainsworth, E.; LaBaer, J. FEBS Journal 2005, 272, 5412.
2, Yu, X; Xu, D.; Cheng, Q. Label-free detection methods for protein microarrays, Proteomics 2006, 6, 5493.
3. Cooper, M. A. Drug Discov. Today 2006, 11, 1068.
4. Wiliets, K. A.; Van Duyne, R. P. Annu. Rev, Phys. Chem. 2007, 58, 267.
5. Phillips, K. S.; Cheng, Q. Anal. Bioanal. Chem. 2007, 387, 1831,
6. Wegner, G. J.; Wark, A. W.; Lee, H. J.; Codner, E.; Sacki, T; Fang, S.; Corn, R. M. Anal. Chem. 2004, 76, 5677.
7. Lee, H. J.; Wark, A. W.: Corn, R. M, Langmuir 2006, 22, 5241.
8, Lee, H. J.; Nedelkov, D.; Corn, R. M. Anal. Chem. 2006, 78, 6504.
9. Lakowicz, J. R. Principles of Fluorescence Spectroscopy, 3rd Edition, Springer, New York, 2006.
10. Taflove, A.; Hagness, S. C. Computational Electrodynamics: The Finite-Difference Time-Domain Method; Artech House: Boston, 2000,
11. Sullivan, D. M. Electromagnetic simulation using the FDTD method, IEEE Press: New York, 2000.
12. Yang P.; Liou, N. K. Opt. Soc. Am. A 1996, 13, 2072.
13. Gray, S. K.; Kupka, T Phy. Rev, B, 2003, 68, 045415.
14. Chang, S.; Gray, S. K.; Schatz, G. C. Opt. Exp. 2005, 13, 3150.
15. Taflove, A.; Brodwin, M. E. IEEE Trans. Microwave Theory and Techniques 1975, 23, 623.
16. Reference Guide for FDTD Solutions™ Release 5.0, 2007, http:/www.lumerical.com/fdtd.
17. Chowdhury, M. H.; Gray, S. K.; Pond, J.; Geddes, C. D.; Asian, K.; Lakowicz, J. R. J. Opt. Soc. Am. B 2007, 24, 2259.
18. Chowdhury, M. H.; Pond, J.; Gray, S. K.; Lakowicz, J. R. J. Phys. Chem. C 2008, 112, 11236.
19. Ray, K.; Badugu, R.; Lakowicz, J. R. J. Phys. Chem. B 2006, 110, 13499.
20. Ray, K.; Badugu, R.; Lakowicz, J. R. J. Phys. Chem, C 2007, 111, 7091.
21. Ray, K.; Badugu, R.; Lakowicz, J. R. Chem. Mat. 2007, 19, 5902.
22. Kaminski, F.; Sandoghdar, V.; Agio, M. J. Comp, and Theor. Nanoscience 2007, 4, 635.
23. Jackson, J. D. Classical Electrodynamics (2nd Ed.); John Wiley & Sons: New York, 1975.
24. Lakowicz, I. R.; Ray, K.; Chowdhury, M.; Szmacinaski, H.; Fu, Y.; Zhang, J.; Nowaczyk, K. Analyst, 2008, DOI: 10.1039/b802918k.
25. Ray, K.; Chowdhury, M.; Lakowicz, J. R. Anal, Chem. 2007, 79, 6480.
26. Lakowicz, J. R. Anal. Anal. Biochem. 2001, 298, 1-24.
27. Lakowicz, J. R. Anal. Biochem. 2005, 337, 171-194.
28. Ray, K; Badugu, R.; Lakowicz, J. R. J. Am. Chem. Soc. 2006, 128, 8998.
29. Shadowitz, A. The Electromagnetic Field; Dover; New York, 1988.

While the invention has been described in terms of its preferred embodiments, those skilled in the art will recognize that the invention can, be practiced with modification within the spirit and scope of the appended claims. Accordingly, the present invention should not be limited to the embodiments as described above, but should further include all modifications and equivalents thereof within the spirit and scope of the description provided herein.

We claim:

1. A label-free detecting system for detecting a biomolecule that has intrinsic fluorescence, comprising:
a sensing surface comprising at least one nanostructured metal;
a source of electromagnetic radiation that is used to interrogate said sensing surface; and
a detector for detecting the presence or absence of said biomolecule in a composition based on changes in intrinsic fluorescence of said biomolecule that occur when said biomolecule is in close proximity to said nanostructured metal on said sensing surface.

2. The label-free detecting system of claim 1 further comprising s substrate, said at least one nanostructured metal being deposited on said substrate.

3. The label-fee detecting system of claim 2 wherein said substrate is a dielectric substrate.

4. The label-free detecting system of claim 3 wherein said dielectric substrate is selected from the group consisting of silica, silicon, quartz, plastic, silicon nitride, metallic oxides with either no or low fluorescence in the UV wavelength range, and glass.

5. The label-free detecting system of claim 2 wherein said substrate transmits electromagnetic radiation therethrough.

6. The label-free detecting system of claim 2 wherein said substrate is selected from the consisting of a well, a plate, a tube, a wire, and a bead.

7. The label-free detecting system of claim 2 wherein said at least one nanostructured metal includes one or more of aluminum, silver, platinum and gold.

8. The label-free detecting system of claim 2 wherein said at least one nanostructured metal is in a form selected from the group consisting of a nanostructured film, a metal colloid, one or more nanoparticles deposited on a support, a nanoparticle dimer, a nanoparticle cluster, a nanoparticle array on a support, and patterned nanoholes in a layer of continuous metal film.

9. The label-free detecting system of claim 2 wherein said at least one nanostructured metal is present on said substrate in the form of a patterned array of metal nanoparticles, nanoholes, or metal surfaces.

10. The label free-free detecting system of claim 2 wherein said sensing surface includes a grating which is part of one or more of said substrate and said at least one nanostructured metal.

11. The label-free detecting system of claim 2 further comprising a capture molecule associated with said sensing surface which binds to or interacts with said biomolecule.

12. The label-free detecting system of claim 11, wherein said capture molecule is selected from the group consisting of an antibody or antigen.

13. The label-free detecting system of claim 11 wherein said capture molecule is or includes one or more of proteins, peptides, nucleic acids, carbohydrates, cofactors, metal ions, small molecule candidates, enzyme substrates, inhibitors, agonists, antagonists, and ligands.

14. The label-free detecting system of claim 11 wherein said capture molecule is associated with said sensing surface using a linking moiety which spaces said capture molecule away from said substrate.

15. The label-free detecting system of claim 11 wherein said capture molecule, after binding or interacting with said biomolecule, maintains, for a period of time, said biomolecule in close proximity to said at least one nano-structured metal.

16. The label-free detecting system of claim 15 wherein said close proximity ranges between 2 to 50 nm.

17. The label-free detecting system of claim 1 wherein said at least one nanostructured metal includes one or more of aluminum, silver, platinum and gold.

18. The label-free detecting system claim 1 wherein said at least one nanostructured metal is in a form selected from the group consisting of a grating, a nanostructured film, a metal colloid, one or more nanoparticles deposited on a support, a nanoparticle dimer, a nanoparticle cluster, a nanoparticle array on a support, and patterned nanoholes in a layer of continuous metal film.

19. The label-free detecting system of claim 1 wherein said at least one nanostructured metal includes nanoparticles of a size ranging from 20 nm to 100 nm.

20. The label-free detecting system of claim 1 wherein said at least one nanostructured metal includes nanostructured silver particles with a size ranging from 40 nm to 100 nm.
